(19) United States
(12) Patent Application Publication
(10) Pub. No.: US 2023/0414270 A1
(43) Pub. Date: Dec. 28, 2023

(54) DEVICES FOR THE DELIVERY OF PULSED ELECTRIC FIELDS IN THE TREATMENT OF CARDIAC TISSUE

(71) Applicant: Galvanize Therapeutics, Inc., Redwood City, CA (US)

(72) Inventors: Isidro GANDIONCO, Fremont, CA (US); Kirby CHIANG, Mountain View, CA (US); Curt Robert EYSTER, Rancho Cucamonga, CA (US)

(73) Assignee: Galvanize Therapeutics, Inc., Redwood City, CA (US)

(21) Appl. No.: 18/243,604

(22) Filed: Sep. 7, 2023

Related U.S. Application Data
(63) Continuation of application No. PCT/US22/19719, filed on Mar. 10, 2022.
(60) Provisional application No. 63/159,331, filed on Mar. 10, 2021.

(51) Int. Cl. A61B 18/14

(52) U.S. Cl. CPC A61B 18/14; A61B 2018/00267

(57) ABSTRACT

Devices, systems and methods are provided for treating conditions of the heart, particularly the occurrence of arrhythmias, more particularly atrial fibrillation, atrial flutter, ventricular tachycardia, to name a few. The devices, systems and methods deliver therapeutic pulsed electric field energy to portions the heart to provide tissue modification, such as to the entrances to the pulmonary veins in the treatment of atrial fibrillation. Such tissue modification creates a conduction block within the tissue to prevent the transmission of aberrant electrical signals. Generally, the tissue modification systems include a specialized catheter, a high voltage waveform generator and at least one distinct energy delivery algorithm. Example embodiments of specialized catheter designs are provided and include a variety of delivery types including focal delivery, “one-shot” delivery and various possible combinations.

### CROSS-REFERENCE TO RELATED APPLICATIONS

This application is a continuation of PCT Application No. PCT/US22/19719, filed Mar. 10, 2022, which claims priority to and the benefit of U.S. Patent Application No. 63/159,331, filed Mar. 10, 2021, the disclosures of which are fully incorporated herein.

### BACKGROUND

Therapeutic energy can be applied to the heart and vasculature for the treatment of a variety of conditions, including atherosclerosis (particularly in the prevention of restenosis following angioplasty) and arrhythmias, such as atrial fibrillation. Atrial fibrillation is the most common sustained cardiac arrhythmia, and severely increases the risk of mortality in affected patients, particularly by causing stroke. In this phenomenon, the heart is taken out of normal sinus rhythm due to the production of erroneous electrical impulses. Atrial fibrillation is thought to be initiated in the myocardial sleeves of the pulmonary veins (PVs) due to the presence of automaticity in cells within the myocardial tissue of the PVs. Pacemaker activity from these cells is thought to result in the formation of ectopic beats that initiate atrial fibrillation. PVs are also thought to be important in the maintenance of atrial fibrillation because the chaotic architecture and electrophysiological properties of these vessels provides an environment where atrial fibrillation can be perpetuated. Thus, destruction or removal of these aberrant pacemaker cells within the myocardial sleeves of the PVs has been a goal and atrial fibrillation is often treated by delivering therapeutic energy to the pulmonary veins. However, due to reports of PV stenosis, the approach has been conventionally modified to one that targets PV antra to achieve conduction block between the PVs and the left atrium. The PV antra encompass, in addition to the pulmonary veins, the left atrial roof and posterior wall and, in the case of the right pulmonary vein antra, a portion of the interatrial septum. In some instances, this technique offers a higher success rate and a lower complication rate compared with pulmonary vein ostial isolation.

Thermal ablation therapies, especially radiofrequency (RF) ablation, are currently the “gold standard” to treat symptomatic atrial fibrillation by localized tissue necrosis. Typically, RF ablation is used to create a ring of ablation lesions around the outside of the ostium of each of the four pulmonary veins. RF current causes desiccation of tissue by creating a localized area of heat that results in discrete coagulation necrosis. The necrosed tissue acts as a conduction block thereby electrically isolating the veins.

Despite the improvements in reestablishing sinus rhythm using available methods, both success rate and safety are limited. RF ablation continues to present multiple limitations including long procedure times to perform pulmonary vein isolation with RF focal catheters, potential gaps in ablation patterns due to point-by-point ablation technique with conventional RF catheters, difficulty in creating and confirming transmural ablation lesions, char and/or gas formation at the catheter tip-tissue interface due to high temperatures, which may lead to thrombus or emboli during ablation, and thermal damage to collateral extracardiac structures, which include pulmonary vein stenosis, phrenic nerve injury, esophageal injury, atrio-esophageal fistula, peri-esophageal vagal injury, perforations, thromboembolic events, vascular complications, and acute coronary artery occlusion, to name a few. These limitations are primarily attributed to the continuous battle clinicians have faced balancing effective therapeutic dose with inappropriate energy delivery to extracardiac tissue.

Thus, while keeping the technique in clinical practice, safer and more versatile methods of removing abnormal tissue have been used, including irreversible electroporation (IRE), a non-thermal therapy based on the unrecoverable permeabilization of cell membranes caused by particular short pulses of high voltage energy. IRE has been found to be tissue-specific, triggering apoptosis rather than necrosis, and safer for the structures adjacent the myocardium. However, thus far, the success of these IRE methodologies has been heterogeneous. In some instances, the delivery of IRE energy has resulted in incomplete block of the aberrant electrical rhythms. This may be due to a variety of factors, such as irregularity of treatment circumferentially around the pulmonary veins, lack of transmural delivery of energy or other deficiencies in the delivery of energy. In either case, atrial fibrillation is not sufficiently treated or atrial fibrillation recurs at a later time. Therefore, improvements in atrial fibrillation treatment are desired. Such treatments should be safe, effective, and lead to reduced complications. At least some of these objectives will be met by the systems, devices and methods described herein.

### SUMMARY

Described herein are embodiments of apparatuses, systems and methods for treating target tissue, particularly cardiac tissue. Likewise, the invention relates to the following numbered clauses:
1. A device for delivering energy to cardiac tissue of a patient comprising:
a shaft having a proximal end and a distal end, wherein the shaft has an outer diameter; and
an energy delivery body disposed along the distal end of the shaft, wherein the energy delivery body is transitionable between a collapsed configuration and an expanded configuration, wherein the expanded configuration has an outer diameter that is less than or equal to 6 times the outer diameter of the shaft, wherein the energy delivery body is configured to be positioned against the cardiac tissue in the expanded configuration so as to deliver energy to the cardiac tissue.
2. A device as in claim 1, wherein the energy is pulsed electric field energy and wherein the device is configured to deliver the pulsed electric field energy to the cardiac tissue.
3. A device as in any of the above claims, wherein the energy delivery body comprises a plurality of wires configured to deliver the energy.
4. A device as in claim 3, wherein the plurality of wires comprises a plurality of splines.
5. A device as any of claims 3-4, wherein the plurality of wires is comprised of shape memory material so that the energy delivery body is transitionable by release from a sheath that constrains the plurality of wires so that such release allows the plurality of wires to move toward the expanded configuration.
6. A device as in claim 5, wherein the energy delivery body is free of a central shaft when in the expanded configuration.
7. A device as in claim 5, wherein the plurality of splines form a hollow rounded cage.
8. A device as in claim 3, wherein the plurality of wires comprises a mesh.
9. A device as in claim 3, wherein the plurality of wires comprises a plurality of loops.
10. A device as in any of claims 3-9, wherein the plurality of wires is energizable in unison so as to function in a monopolar fashion.
11. A device as in any of claims 3-10, wherein the plurality of wires has a convex distal face.
12. A device as in any of claims 3-11, wherein the energy delivery body includes a distal tip configured to deliver the energy.
13. A device as in claim 12, wherein the distal tip and the plurality of wires are energizable in unison so as to function in a monopolar fashion.
14. A device as in any of claims 3-13, wherein a proximal portion of the plurality of wires is insulated so as to direct the energy toward a distal direction.
15. A device as in any of the above claims, further comprising a plurality of irrigation ports, wherein the device is configured so as to direct fluid through the irrigation ports in a manner that creates turbulent flow of the fluid within the energy delivery body.
16. A device as in claim 15, wherein the plurality of irrigation ports are disposed near a proximal end of the energy delivery body.
17. A device as in any of claims 15-16, further comprising one or more irrigation lumens which direct the fluid through the plurality of irrigation ports.
18. A device as in claim 17, wherein the one or more irrigation lumens is less than the plurality of irrigation ports.
19. A device as in any of the above claims, wherein the expanded configuration has an outer diameter that is 3-6 times the outer diameter of the shaft.
20. A device as in any of the above claims, wherein the expanded configuration has an outer diameter that is 8-15 mm.
21. A device as in any of the above claims, wherein the energy delivery body includes a sensing electrode.
22. A device as in claim 21, wherein the sensing electrode is positioned so as to avoid contact with the cardiac tissue.
23. A device as in claim 22, wherein the energy delivery body comprises a plurality of splines forming a rounded cage and wherein the sensing electrode is disposed within the rounded cage.
24. A device as in any of the above claims, wherein the shaft includes one or more ring electrodes.
25. A device as in any of the above claims, further comprising one or more electrodes that communicate with an electrophysiological mapping system.
26. A device as in any of the above claims, further comprising a steering mechanism configured to bend the energy delivery body in relation to the shaft.
27. A device as in any of the above claims, further comprising a steering mechanism configured to bend the distal end of the shaft away from its longitudinal axis.
28. A device for treating cardiac tissue of a patient comprising:
a shaft having a proximal end and a distal end; and
an energy delivery body disposed along the distal end of the shaft, wherein the energy delivery body is configured to be positioned against the cardiac tissue, and wherein the energy delivery body is electrically couplable with a generator so as to deliver pulsed electric field energy to the cardiac tissue.
29. A catheter as in claim 28, wherein the energy delivery body comprises one or more loops shaped from wire.
30. A catheter as in any of claims 28-29, wherein the at least one electrode comprises one or more loops arranged to form a continuous rim that is configured to contact the cardiac tissue.
31. A catheter as in claim 30, wherein the continuous rim has a closed shape having a diameter of 8-15 mm.
32. A catheter as in claim 30, wherein the continuous rim has a closed shape having a diameter smaller than an internal diameter of a pulmonary vein.
33. A catheter as in claim 30, wherein the continuous rim has a closed shape configured to mate with an opening of a pulmonary vein so as to create a continuous lesion around the opening of the pulmonary vein.
34. A catheter as in any of claims 30-33, wherein the continuous rim forms a closed shape having an adjustable diameter.
35. A catheter as in any of claims 30-34, further comprising an electrode disposed along the distal end of the shaft so as to be able to contact the cardiac tissue when the continuous rim is positioned against the cardiac tissue and pressure is applied.
36. A catheter as in claim 35, wherein the electrode is disposed along the distal end of the shaft so as to disengage contact with the cardiac tissue when pressure is released from the continuous rim.
37. A catheter as in any of claims 28-36, wherein at least a portion of the energy delivery body is configured to at flex when the energy delivery body is positioned against the cardiac tissue pressure and pressure is applied.
38. A catheter as in claim 28, wherein the energy delivery body comprises one or more loops forming a convex distal face.
39. A catheter as in claim 38, wherein the convex distal face is configured to seat against an inlet of a pulmonary vein.
40. A catheter as in claim 39, wherein at least a portion of the convex distal face is configured to seat within the pulmonary vein.
41. A catheter as in claim 28, wherein the energy delivery body comprises one or more loops forming a concave distal face.
42. A catheter as in claim 41, wherein the one or more loops comprise two pairs of loops, wherein each pair of loops comprises a smaller loop within a larger loop.
43. A catheter as in claim 42, wherein the shaft has a longitudinal axis and wherein each of the two pairs of loops extend in opposite directions from the longitudinal axis.
44. A catheter as in claim 28, wherein the energy delivery body comprises a single paddle shaped electrode having a narrower shape near the shaft and a wider shape extending away from the shaft.

45. A catheter as in claim 44, wherein the wider shape has a hammerhead shape.
46. A catheter as in claim 28, wherein the energy delivery body is transitionable between a collapsed configuration and an expanded configuration, wherein the expanded configuration has an outer diameter that is less than or equal to 6 times the outer diameter of the shaft, wherein the energy delivery body is configured to be positioned against the cardiac tissue in the expanded configuration so as to deliver energy to the cardiac tissue.
47. A device for delivering energy to cardiac tissue of a patient comprising:
a shaft having a proximal end and a distal end; and
an energy delivery body disposed along the distal end of the shaft, wherein the energy delivery body is comprised of a plurality of shape-memory splines and is transitionable between a collapsed configuration and an expanded configuration, wherein in the expanded configuration the plurality of shape-memory splines form a convex distal face positionable against the cardiac tissue so as to deliver energy to the cardiac tissue.
48. A device as in claim 47, wherein the plurality of shape-memory splines form a rounded cage having the convex distal face in the expanded configuration.
49. A device as in claim 48, wherein the rounded cage is supported solely by the plurality of splines.
50. A device as in claim 49, wherein the energy delivery body includes a distal tip electrode and wherein the plurality of splines supports a tip electrode wire extending from the distal tip electrode to the shaft and is otherwise hollow.
51. A device as in any of claims 48-50, wherein the rounded cage is flexible so as to deform upon positioning against the cardiac tissue.
52. A device as in any of claims 48-51, wherein the rounded cage is flexible so as to at least partially flatten upon positioning against the cardiac tissue.
53. A device as in any of claims 48-52, wherein the convex distal face is configured to have a footprint of 8-15 mm when positioned against the cardiac tissue so as to deliver energy to the cardiac tissue.
54. A device as in any of the above claims, wherein the plurality of splines are energizable in unison to function in a monopolar manner.
55. A device as in any of the above claims, wherein the energy delivery body includes a distal tip electrode disposed along the convex distal face.
56. A device as in claim 55, wherein the distal tip electrode is independently energizable.
57. A device as in any of the above claims, wherein at least a portion of the energy delivery body is insulated so as to direct the energy through the convex distal face.
58. A device as in any of the above claims, further comprising at least one irrigation lumen and a plurality of irrigation ports.
59. A device as in claim 58, wherein the at least one irrigation lumen comprises a number of irrigation lumens that is less than plurality of irrigation ports.
60. A device as in claim 48, wherein the energy delivery body is electrically couplable with a generator so as to deliver pulsed electric field energy to the cardiac tissue.
61. A system for delivering energy to cardiac tissue of a patient comprising:
a treatment catheter comprising
   a shaft having a proximal end and a distal end, wherein the shaft has an outer diameter, and
   an energy delivery body disposed along the distal end of the shaft, wherein the energy delivery body is transitionable between a collapsed configuration and an expanded configuration, wherein the expanded configuration has an outer diameter that is less than or equal to 6 times the outer diameter of the shaft, wherein the energy delivery body is configured to be positioned against the cardiac tissue in the expanded configuration so as to deliver energy to the cardiac tissue; and
a generator electrically couplable to the treatment catheter, wherein the generator includes at least one energy delivery algorithm configured to provide an electric signal of pulsed electric field energy deliverable through the energy delivery body.
62. A system as in claim 61, wherein the pulsed electric field energy is below a threshold for inducing coagulative thermal damage.
63. A system for treating cardiac tissue of a patient comprising:
a treatment device comprising
   a shaft having a proximal end and a distal end, and
   an energy delivery body disposed along the distal end of the shaft, wherein the energy delivery body is configured to be positioned against the cardiac tissue, and wherein the energy delivery body is electrically couplable with a generator so as to deliver pulsed electric field energy to the cardiac tissue; and
a generator electrically couplable to the treatment catheter, wherein the generator includes at least one energy delivery algorithm configured to provide an electric signal of pulsed electric field energy deliverable through the energy delivery body.
64. A system as in claim 63, wherein the pulsed electric field energy is below a threshold for inducing coagulative thermal damage.
65. A system for delivering energy to cardiac tissue of a patient comprising:
a treatment catheter comprising
   a shaft having a proximal end and a distal end, and
   an energy delivery body disposed along the distal end of the shaft, wherein the energy delivery body is comprised of a plurality of shape-memory splines and is transitionable between a collapsed configuration and an expanded configuration, wherein in the expanded configuration the plurality of shape-memory splines form a convex distal face positionable against the cardiac tissue so as to deliver energy to the cardiac tissue; and
a generator electrically couplable to the treatment catheter, wherein the generator includes at least one energy delivery algorithm configured to provide an electric signal of pulsed electric field energy deliverable through the energy delivery body.
66. A system as in claim 65, wherein the pulsed electric field energy is below a threshold for inducing coagulative thermal damage.
67. A method of treating a patient comprising:
advancing a distal end of a catheter into a heart of the patient, wherein the catheter has an energy delivery body disposed along its distal end;
positioning a return electrode remote from the distal end of the catheter;
positioning at least a portion of the energy delivery body at a first location along an area of cardiac tissue;
delivering pulsed electric field energy through the at least one electrode monopolarly so that the pulsed electric field energy is directed through the cardiac tissue to the return electrode; and
repeatedly re-positioning the at least a portion of the energy delivery body at one or more additional locations along the area of cardiac tissue so as to create a continuous lesion.
68. A method as in claim 67, wherein the first location and the one or more additional locations create a closed shape around a pulmonary vein of the heart.
69. A method as in claim 67, wherein the continuous lesion has a depth sufficient to block conduction between the pulmonary vein and a remainder of the heart.
70. A method as in claim 68, wherein the first location and the one or more additional locations create a linear shape having a depth sufficient to block conduction.
71. A method as in claim 67, wherein the energy delivery body comprises one or more loops arranged to form a continuous rim, and wherein positioning at least a portion of the energy delivery body comprises positioning the continuous rim.
72. A method as in claim 67, wherein the energy delivery body comprises one or more loops shaped from a wire.
These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an embodiment of a tissue modification system.

FIGS. 2A-2B illustrates embodiments of a treatment catheter configured to deliver focal therapy.

FIG. 3 illustrates a portion of the heart showing a cut-away of the right atrium and left atrium with a treatment catheter positioned therein.

FIG. 4 illustrates the repeated application of energy in point by point fashion around the left inferior pulmonary vein with the use of the treatment catheter to create a circular treatment zone.

FIG. 5 illustrates an embodiment of a waveform of a signal prescribed by an energy delivery algorithm.

FIG. 6 illustrates an example waveform prescribed by an energy delivery algorithm wherein the waveform has voltage imbalance.

FIG. 7 illustrates further examples of waveforms having unequal voltages.

FIG. 8 illustrates examples of waveforms having unequal pulse widths.

FIG. 9 illustrates an example waveform prescribed by another energy delivery algorithm wherein the waveform is monophasic, a special case of imbalance whereby there is only a positive or only a negative portion of the waveform.

FIG. 10 illustrates further examples of waveforms having monophasic pulses.

FIG. 11 illustrates examples of waveforms having phase imbalances.

FIG. 12 illustrates an example waveform prescribed by another energy delivery algorithm wherein the pulses are sinusoidal in shape rather than square.

FIGS. 13A-13C illustrate an embodiment of a treatment catheter configured for focal delivery that optionally covers a larger area of tissue than a cylindrically shaped delivery electrode typically found on conventional RF catheters.

FIGS. 14A-14D illustrate delivery of the embodiment of the catheter of FIGS. 13A-13C.

FIG. 15 provides a visual illustration of example end effectors adjacent to each other in contact with tissue.

FIG. 16 illustrates another embodiment of a treatment catheter configured for focal delivery, that optionally covers a larger area of tissue than a cylindrically shaped delivery electrode typically found on conventional RF catheters, or for one shot therapy.

FIG. 17 illustrates the embodiment of FIG. 16 positioned against a laboratory benchtop model of the entrance to the pulmonary vein.

FIGS. 18A-18B illustrate the delivery electrode deployed from a delivery sheath so that the electrodes extend substantially perpendicular to a longitudinal axis of the delivery sheath.

FIG. 19 illustrates further extension of the electrodes of FIGS. 18A-18B from the sheath which allows the petal shaped electrodes to curve downward so that the external rims of the electrodes are disposed proximally of the distal tip of the sheath.

FIG. 20 illustrates still further deployment of the electrodes of FIGS. 18A-18B which exaggerates this shape wherein the sides are allowed to bend or arc even further.

FIG. 21 illustrates an embodiment of a treatment catheter configured for focal delivery rather than one shot delivery or combination delivery.

FIG. 22 illustrates an embodiment of the treatment catheter wherein the delivery electrode comprises a plurality of trowel shaped electrodes extending from a shaft.

FIG. 23 illustrates a side view of an embodiment of a treatment catheter similar to that of FIGS. 21-22.

FIG. 24 illustrates another embodiment of a treatment catheter comprising a plurality of trowel shaped electrodes extending from a shaft, however here the base is a free end and the tip is attached to the catheter by a support.

FIG. 25 illustrates an embodiment of a treatment catheter wherein the delivery electrode comprises a single petal, paddle or loop shaped electrode having a narrower shape near the shaft and a larger, wider shape extending away from the shaft.

FIGS. 26A-26B illustrate an embodiment of a treatment catheter having a paddle shaped delivery electrode wherein the delivery electrode comprises a single hammerhead paddle shaped electrode having a narrower shape near the shaft and a wider, hammerhead shape distal from the shaft.

FIGS. 27-30 illustrates another embodiment of a treatment catheter.

FIGS. 31A-31D illustrate an embodiment of a treatment catheter configured for one shot delivery rather than focal delivery.

FIGS. 32A-32C illustrate another embodiment of a treatment catheter.

FIGS. 33A-33D illustrate yet another embodiment of a treatment catheter.

FIG. 34 illustrates an embodiment of a treatment catheter having an energy delivery body comprised of a plurality of splines.

FIG. 35 provides a side view of the embodiment of the treatment catheter of FIG. 34.

FIG. 36 illustrates a bottom view of the treatment catheter of FIGS. 34-35.

FIG. 37 provides another perspective view of the embodiment of FIG. 34.

FIG. 38 provides a close-up illustration of a portion of the treatment catheter of FIG. 34 within the distal end of the shaft.

FIG. 39A provides an expanded illustration of elements comprising this embodiment of the treatment catheter of FIG. 34.

FIG. 39B illustrates the treatment catheter of FIG. 39A in its unexpanded state.

### DETAILED DESCRIPTION

Devices, systems and methods are provided for treating conditions of the heart, particularly the occurrence of arrhythmias, more particularly atrial fibrillation, atrial flutter, ventricular tachycardia, Wolff-Parkinson-White syndrome, and/or atrioventricular nodal reentry tachycardia, to name a few. The devices, systems and methods deliver therapeutic energy to portions the heart to provide tissue modification, such as to the entrances to the pulmonary veins in the treatment of atrial fibrillation. Targeted specific anatomic locations include the superior vena cava, inferior vena cava, right pulmonary vein, left pulmonary vein, right atrium, right atrial appendage, left atrium, left atrial appendage, right ventricle, left ventricle, right ventricular outflow tract, left ventricular outflow tract, ventricular septum, left ventricular summit, regions of myocardial scar, myocardial infarction border zones, myocardial infarction channels, ventricular endocardium, ventricular epicardium, papillary muscles and the Purkinje system, to name a few. Treatments are delivered at isolated sites or in a connected series of treatments. Types of treatment include the creation of left atrial roof line, left atrial posterior/inferior line, posterior wall isolation, lateral mitral isthmus line, septal mitral isthmus line, left atrial appendage, right sided cavotricuspid isthmus (CTI), pulmonary vein isolation, superior vena cava isolation, vein of Marshall, lesion creation using Complex Fractionated Atrial Electrograms (CFAE), lesion creation using Focal Impulse and Rotor Modulation (FIRM), and targeted ganglia ablation. Such tissue modification creates a conduction block within the tissue to prevent the transmission of aberrant electrical signals. The devices, systems and methods are typically used in an electrophysiology lab or controlled surgical suite equipped with fluoroscopy and advanced ECG recording and monitoring capability. An electrophysiologist (EP) is typically the intended primary user of the system. The electrophysiologist will be supported by a staff of trained nurses, technicians, and potentially other electrophysiologists. Generally, the tissue modification systems include a specialized catheter, a high voltage waveform generator and at least one distinct energy delivery algorithm. Additional accessories and equipment may be utilized. Example embodiments of specialized catheter designs are provided herein and include a variety of delivery types including focal delivery, “one-shot” delivery and various possible combinations. For illustration purposes a simplified design is provided when describing the overall system. Such a simplified design provides monopolar focal therapy. However, it may be appreciated that a variety of other embodiments are also provided.

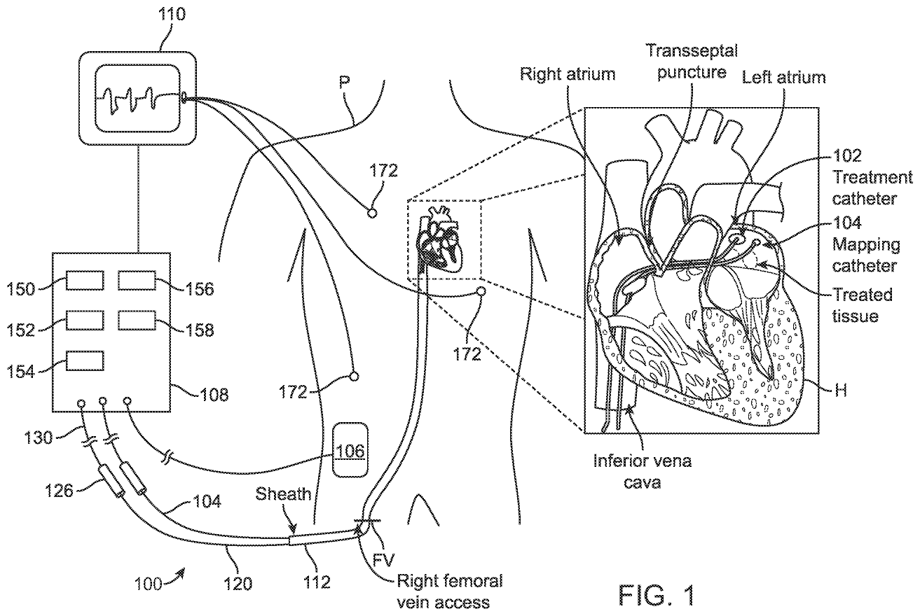

FIG. 1 illustrates an embodiment of a tissue modification system 100 comprising a treatment catheter 102, a mapping catheter 104, a return electrode 106, a waveform generator 108 and an external cardiac monitor 110. In this embodiment, the heart is accessed via the right femoral vein FV by a suitable access procedure, such as the Seldinger technique. Typically, a sheath 112 is inserted into the femoral vein FV which acts as a conduit through which various catheters and/or tools may be advanced, including the treatment catheter 102 and mapping catheter 104. It may be appreciated that in some embodiments, the treatment catheter 102 and mapping catheter 104 are combined into a single device. As illustrated in FIG. 1, the distal ends of the catheters 102, 104 are advanced through the inferior vena cava, through the right atrium, through a transseptal puncture and into the left atrium so as to access the entrances to the pulmonary veins. The mapping catheter 104 is used to perform cardiac mapping which refers to the process of identifying the temporal and spatial distributions of myocardial electrical potentials during a particular heart rhythm. Cardiac mapping during an aberrant heart rhythm aims at elucidation of the mechanisms of the heart rhythm, description of the propagation of activation from its initiation to its completion within a region of interest, and identification of the site of origin or a critical site of conduction to serve as a target for treatment. Once the desired treatment locations are identified, the treatment catheter 102 is utilized to deliver the treatment energy.

In this embodiment, the proximal end of the treatment catheter 102 is electrically connected with the waveform generator 108, wherein the generator 108 is software-controlled with regulated energy output that creates high frequency short duration energy delivered to the catheter 102. It may be appreciated that in various embodiments the output is controlled or modified to achieve a desired voltage, current, or combination thereof. In this embodiment, the proximal end of the mapping catheter 104 is also electrically connected with the waveform generator 108 and the electronics to perform the mapping procedure are included in the generator 108. However, it may be appreciated that the mapping catheter 104 may alternatively be connected with a separate external device having the capability of providing the mapping procedure, such as electroanatomic mapping (EAM) systems (e.g. CARTO® systems by Biosense Webster/Johnson & Johnson, EnSite™ systems by St. Jude Medical/Abbott, KODEX-EPD system by Philips, Rhythmia HDX™ system by Boston Scientific). Likewise, in some embodiments, a separate mapping catheter 104 is not used and the mapping features are built into the catheter 102.

In this embodiment, the generator 108 is connected with an external cardiac monitor 110 to allow coordinated delivery of energy with the cardiac signal sensed from the patient P. The generator synchronizes the energy output to the patient's cardiac rhythm. The cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. Typically, a footswitch allows the user to initiate and control the delivery of the energy output. The generator user interface (UI) provides both audio and visual information to the user regarding energy delivery and the generator operating status.

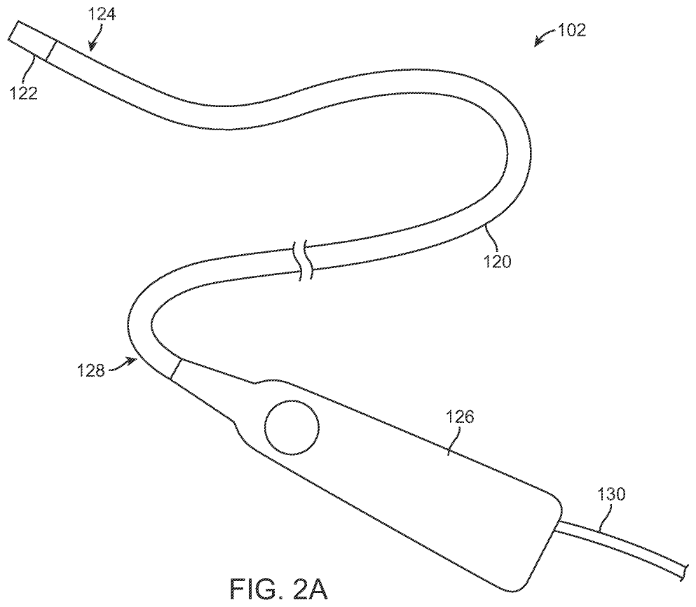

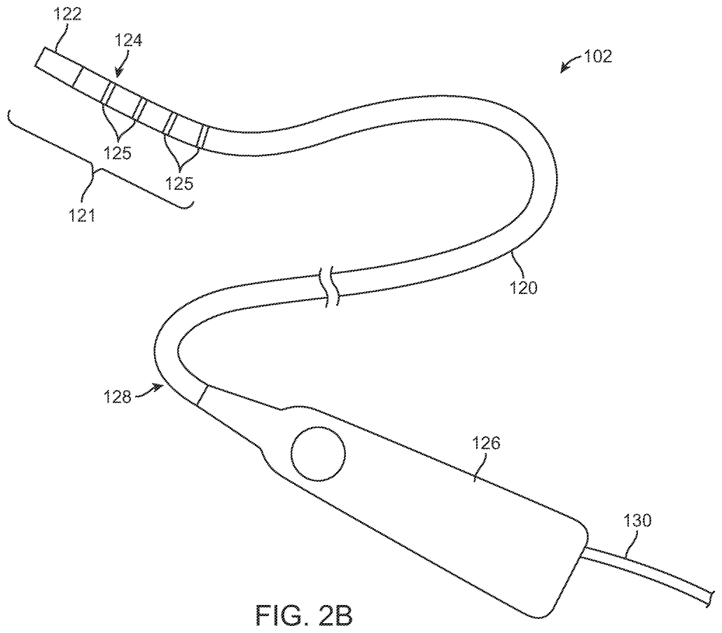

In this embodiment, the treatment catheter 102 is designed to be monopolar, wherein the distal end of the catheter 108 has as a delivery electrode 122 and the return electrode 106 is positioned upon the skin outside the body, typically on the thigh, lower back or back. FIG. 2A illustrates an embodiment of a treatment catheter 102 configured to deliver focal therapy. In this embodiment, the catheter 102 comprises an elongate shaft 120 having a delivery electrode 122 near its distal end 124 and a handle 126 near its proximal end 128. The delivery electrode 122 is shown as a “solid tip” electrode having a cylindrical shape with a distal face having a continuous surface. In some embodiments, the cylindrical shape has a diameter across its distal face of approximately 2-3 mm and a length along the shaft 120 of approximately lmm, 2 mm, 1-2 mm, 3 mm, 4 mm, 3-4 mm, 5 mm, 6 mm, 7 mm, 8 mm, 9 mm, 10 mm, etc. It may be appreciated that such electrodes are typically hollow yet are referred to as solid due to visual appearance. In some embodiments, the catheter 102 has an overall length of 50-150 cm, preferably 100-125 cm, more preferably 110-115 cm. Likewise, in some embodiments, it has a 7 Fr outer diameter 3-15 Fr, preferably 4-12 Fr, more preferably 7-8.5 Fr. It may be appreciated that in some embodiments, the shaft 120 has a deflectable end portion 121 and optionally the deflectable end portion 121 may have a length of 50-105 mm resulting in curves with diameters ranging from approximately 15 to 55 mm. Deflection may be achieved by a variety of mechanisms including a pull-wire which extends to the handle 126. Thus, the handle 126 is used to manipulate the catheter 102, particularly to steer the distal end 124 during delivery and treatment. Energy is provided to the catheter 102, and therefore to the delivery electrode 122, via a cable 130 that is connectable to the generator 108.

Pulsed electric fields (PEFs) are provided by the generator 108 and delivered to the tissue through the delivery electrode 122 placed on or near the targeted tissue area. It may be appreciated that in some embodiments, the delivery electrode 122 is positioned in contact with a conductive substance which is likewise in contact with the targeted tissue. Such solutions may include isotonic or hypertonic solutions. These solutions may further include adjuvant materials, such as chemotherapy or calcium, to further enhance the treatment effectiveness both for the focal treatment as well as potential regional infiltration regions of the targeted tissue types. High voltage, short duration biphasic electric pulses are then delivered through the electrode 122 in the vicinity of the target tissue. These electric pulses are provided by at least one energy delivery algorithm 152. In some embodiments, each energy delivery algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

It may be appreciated that in various embodiments the treatment catheter 102 includes a variety of specialized features. For example, in some embodiments, the catheter 102 includes a mechanism for real-time measurement of the contact force applied by the catheter tip to a patient's heart wall during a procedure. In some embodiments, this mechanism is included in the shaft 120 and comprises a tri-axial optical force sensor which utilizes white light interferometry. By monitoring and modifying the applied force throughout the procedure, the user is able to better control the catheter 102 so as to create more consistent and effective lesions.

In some embodiments, the catheter 102 includes one or more additional electrodes 125 (e.g. ring electrodes) positioned along the shaft 120, such as illustrated in FIG. 2B, proximal to the delivery electrode 122. In some embodiments, some or all of the additional electrodes can be used for stimulating and recording (for electrophysiological mapping), so a separate cardiac mapping catheter is not needed when using catheter 102 for lesion creation, or for other purposes such as sensing, etc.

In some embodiments, the catheter 102 includes a thermocouple temperature sensor, optionally embedded in the delivery electrode 122. Likewise, in some embodiments the catheter 102 includes a lumen which may be used for irrigation and/or suction. Typically, the lumen connects with one or more ports along the distal end of the catheter 102, such as for the injection of isotonic saline solution to irrigate or for the removal of, for example, microbubbles.

In some embodiments, the catheter 102 includes one or more sensors that can be used to determine temperature, impedance, resistance, capacitance, conductivity, permittivity, and/or conductance, to name a few. In some embodiments, one or more of the electrodes act as the one or more sensors. In other embodiments, the one or more sensors are separate from the electrodes. Sensor data can be used to plan the therapy, monitor the therapy and/or provide direct feedback via the processor 154, which can then alter the energy-delivery algorithm 152. For example, impedance measurements can be used to determine not only the initial dose to be applied but can also be used to determine the need for further treatment, or not.

Referring back to FIG. 1, in this embodiment the generator 108 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included.

In some embodiments, the generator 108 includes three sub-systems: 1) a high-energy storage system, 2) a high-voltage, medium-frequency switching amplifier, and 3) the system controller, firmware, and user interface. In this embodiment, the system controller includes a cardiac synchronization trigger monitor that allows for synchronizing the pulsed energy output to the patient's cardiac rhythm. The generator takes in alternating current (AC) mains to power multiple direct current (DC) power supplies. The generator's controller can cause the DC power supplies to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only, and systems delivering pulsed electric fields may or may not include additional switching amplifier components.

The user interface 150 can include a touch screen and/or more traditional buttons to allow for the operator to enter patient data, select a treatment algorithm (e.g., energy delivery algorithm 152), initiate energy delivery, view records stored on the storage/retrieval unit 156, and/or otherwise communicate with the generator 108.

In some embodiments, the user interface 150 is configured to receive operator-defined inputs. The operator-defined inputs can include a duration of energy delivery, one or more other timing aspects of the energy delivery pulse, power, and/or mode of operation, or a combination thereof. Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination thereof.

As mentioned, in some embodiments the system 100 also includes a mechanism for acquiring an electrocardiogram (ECG), such as an external cardiac monitor 110, in situations wherein cardiac synchronization is desired. Example cardiac monitors are available from AccuSync Medical Research Corporation and Ivy Biomedical Systems, Inc. In some embodiments, the external cardiac monitor 110 is operatively connected to the generator 108. The cardiac monitor 110 can be used to continuously acquire an ECG signal. External electrodes 172 may be applied to the patient P to acquire the ECG. The generator 108 analyzes one or more cardiac cycles and identifies the beginning of a time period during which it is safe to apply energy to the patient P, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. It will be appreciated that such cardiac synchronization is typically utilized when using monopolar energy delivery, however it may be utilized as part of other energy delivery methods.

In some embodiments, the processor 154, among other activities, modifies and/or switches between the energy-delivery algorithms, monitors the energy delivery and any sensor data, and reacts to monitored data via a feedback loop. In some embodiments, the processor 154 is configured to execute one or more algorithms for running a feedback control loop based on one or more measured system parameters (e.g., current), one or more measured tissue parameters (e.g., impedance), and/or a combination thereof.

The data storage/retrieval unit 156 stores data, such as related to the treatments delivered, and can optionally be downloaded by connecting a device (e.g., a laptop or thumb drive) to a communication port. In some embodiments, the device has local software used to direct the download of information, such as, for example, instructions stored on the data storage/retrieval unit 156 and executable by the processor 154. In some embodiments, the user interface 150 allows for the operator to select to download data to a device and/or system such as, but not limited to, a computer device, a tablet, a mobile device, a server, a workstation, a cloud computing apparatus/system, and/or the like. The communication ports, which can permit wired and/or wireless connectivity, can allow for data download, as just described but also for data upload such as uploading a custom algorithm or providing a software update.

As described herein, a variety of energy delivery algorithms 152 are programmable, or can be pre-programmed, into the generator 108, such as stored in memory or data storage/retrieval unit 156. Alternatively, energy delivery algorithms can be added into the data storage/retrieval unit to be executed by processor 154. Each of these algorithms 152 may be executed by the processor 154.

It may be appreciated that in some embodiments the system 100 includes an automated treatment delivery algorithm that dynamically responds and adjusts and/or terminates treatment in response to inputs such as temperature, impedance at various voltages or AC frequencies, treatment duration or other timing aspects of the energy delivery pulse, treatment power and/or system status.

As mentioned, in some embodiments, the cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. This trigger is within milliseconds of the peak of the R wave (of the ECG QRS complex) to avoid induction of an arrhythmia, which could occur if the energy pulse is delivered on a T wave, and also to ensure that energy delivery occurs at a consistent phase of cardiac contraction. It will be appreciated that such cardiac synchronization is typically utilized when using monopolar energy delivery, however it may be utilized as part of other energy delivery methods.

In this embodiment, the generator 108 is connected with an external cardiac monitor 110 to allow coordinated delivery of energy with the cardiac signal sensed from the patient P.

In some embodiments, the generator 180 receives feedback from the cardiac monitor 110 and responds based on the received information. In some embodiments, the generator 180 receives information regarding the heart rate of the patient and either halts delivery of energy or modifies the energy delivery, such as by selecting a different energy delivery algorithm 152. In some embodiments, the generator 180 halts delivery of energy when the heart rate reaches or drops below a threshold value, such as 30 beats per minute (bpm) or 20 bpm. Optionally, the generator may provide an indicator, such as a visual or auditory indicator, when the heart rate reaches or drops below a lower threshold value, such as providing a flashing yellow light when the heart rate reaches 30 bpm and a solid red light when the heart rate reaches 20 bpm. Such safety measures ensure that the treatment energy is not delivered at an inappropriate time given that low sporadic heart rates may indicate erroneous readings.

In some embodiments, the generator 108 modifies the energy delivery based on the information from the cardiac monitor 110. For example, in some embodiments, energy delivery is provided in a 1:1 ratio when the heart rate is in a predetermined range, such as between 40 bpm and 120 bpm. This involves delivery of PEF energy at the appropriate interval of each heart beat. In some embodiments, the generator 108 modifies the energy delivery if the heart rate exceeds this range, such as if the heart rate exceeds 120 bpm. In some embodiments, the energy delivery is modified to a 2:1 ratio (two heartbeats:one delivery) wherein PEF energy is delivered at the appropriate interval of every other heart beat. It may be appreciated that various ratios of the form m:n (where m and n are integers) may be utilized, such as 3:1, 3:2, 4:1, 4:3 5:1, etc. It may also be appreciated that in some embodiments the heart rate may be paced to achieve a desired heart rate. Such pacing may be provided by a separate or integrated pacemaker. In some embodiments, such pacing is provided by a catheter positioned in the coronary sinus that is used for recording during procedures but is also available for pacing. Such pacing may be triggered by the generator 108 or the cardiac monitor 110.

In some embodiments, the generator 108 halts energy delivery or modifies the energy delivery based on information from other sources, such as from various sensors, including temperature sensors, impedance sensors, contact or contact force sensors, etc. In some embodiments, the generator 108 modifies energy delivery based on sensed temperature (e.g. on the catheter 102, in nearby tissue, in nearby structures, etc.). In some embodiments, energy delivery is modified to a 2:1 ratio, wherein PEF energy is delivered at the appropriate interval of every other heart beat, when the temperature reaches a predetermined threshold value. Such a modification reduces any small thermal effects, thereby reducing sensed temperature. It may be appreciated that various ratios may be utilized, such as 3:1, 3:2, 4:3, 4:1, 5:1, etc.

As mentioned previously, one or more energy delivery algorithms 152 are programmable, or can be pre-programmed, into the generator 108 for delivery to the patient P. The one or more energy delivery algorithms 152 specify electric signals which provide energy delivered to the cardiac tissue which are non-thermal (e.g. below a threshold for thermal ablation; below a threshold for inducing coagulative thermal damage), reducing or avoiding inflammation, and/or preventing denaturation of stromal proteins in the luminal structures. It may be appreciated that the non-thermal energy is also not cryogenic (i.e. it is above a threshold for thermal damage caused by freezing). Thus, the temperature of the target tissue remains in a range between a baseline body temperature (such as 35° C.-37° C. but can be as low as 30° C.) and a threshold for thermal ablation. Thus, targeted ranges of tissue temperature include 30-65° C., 30-60° C., 30-55° C., 30-50° C., 30-45° C., 30-35° C. Thus, lesions in the heart tissue are not created by thermal injury as the temperature of the tissue remains below a threshold for thermal ablation (e.g. 65° C.). In addition, the impedance of the tissue typically remains below a threshold generated by thermal ablation. Charring and thermal injury of tissue changes the conductivity of the heart tissue. This increase in impedance/reduction in conductivity often indicates thermal injury and reduces the ability of the tissue to receive further energy. In some instances, the impedance of the system circuit from the cathode to the anode remains in the range of 25-250Ω, or 50-200Ω during delivery of PEF energy. In general, the algorithms 152 are tailored to affect tissue to a pre-determined depth and/or volume and/or to target specific types of cellular responses to the energy delivered. However, it may be appreciated that the pulsed electric field energy described herein may be utilized more liberally than other types of energy, such as those that cause thermal injury, without negative effects. For instance, since the energy does not cause thermal injury, tissue can be over-treated to ensure sufficient lesion formation. For example, in a tissue layer that is 2 mm thick, energy sufficient to create a lesion having a depth of 6 mm can be applied to the tissue to ensure a transmural lesion. Typically, the additional energy is dissipated away from nearby critical structures through transverse tissue planes. In particular, the pericardial fluid surrounding the heart serves to dissipate energy, protecting extracardiac structures, such as the esophagus, phrenic nerve, coronary arteries, lungs, and bronchioles, from injury. This is not the case when delivering energy that creates lesions by thermal injury. In those cases, the propagation of conductive thermal energy beyond the targeted myocardial tissue can result in thermal injury to non-targeted extracardiac structures. Excessive thermal injury to the esophagus may result in esophageal ulcers that can degrade to a life-threatening atrio-esophageal fistula. Thermal injury to the phrenic nerve may result in permanent diaphragmatic paralysis leading to permanent shortness of breath and fatigue. Thermal injury to the coronary arteries can result in coronary spasm that can lead to temporary, or even permanent, chest pressure/pain. In addition, thermal lesions in the heart, in the region of the pulmonary veins can lead to pulmonary vein stenosis. Pulmonary vein stenosis is a known complication of radiofrequency ablation near the pulmonary veins in patients with atrial fibrillation. This pathologic process is related to thermal injury to the tissue that induces post-procedure fibrosis and scaring. Stenosis has been described in patients treated with many forms of thermal energy, including radiofrequency energy and cryoablation.

Since the PEF lesions described herein are not created by thermal injury, rates of “false positive” confirmation of electrical conduction blocks are also reduced. Thermal injury may result in acute myocardial edema (i.e. tissue fluid accumulation and swelling). When testing electrical conductivity across an area of thermally ablated tissue, the tissue may appear to block electrical conduction however such blocking may simply be the result of temporary edema. After a period of recovery to allow the swelling to subside, this area of treated tissue will no longer have transmural, non-conduction. In addition, acute edema due to thermal injury also diminishes the ability to re-treat an area of tissue. Once an area of tissue has undergone an amount of thermal injury, the resulting edema changes the resistive and conductive thermal properties of the tissue. Therefore, effects similar to the initial response in the tissue are difficult to obtain. Thus, any attempted re-treatment is less effective both acutely and chronically. These issues are avoided with the delivery of the energy described herein.

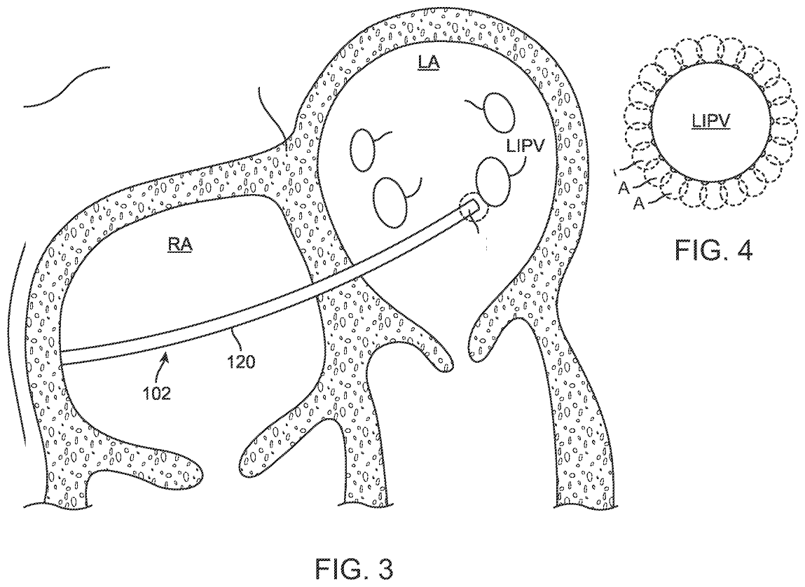

FIG. 3 illustrates a portion of the heart H showing a cut-away of the right atrium RA and left atrium LA in the treatment of atrial fibrillation. The largest pulmonary veins are the four main pulmonary veins (right superior pulmonary vein RSPV, right inferior pulmonary vein RIPV, left superior pulmonary vein LSPV and left inferior pulmonary vein LIPV), two from each lung that drain into the left atrium LA of the heart H. Each pulmonary vein is linked to a network of capillaries in the alveoli of each lung and bring oxygenated blood to the left atrium LA. The left atrial musculature extends from the left atrium LA and envelopes the proximal pulmonary veins. The superior veins, which have longer muscular sleeves, have been reported to be more arrhythmogenic than the inferior veins. In general, the length of the pulmonary vein sleeves varies between 13 mm and 25 mm. Pulmonary vein morphology has been reported to influence arrhythmogenesis. Likewise, cellular electrophysiology and other aspects of the pulmonary veins are associated with arrhythmogenesis and propagation.

A variety of methods are used to determine which tissue is targeted for treatment, such as anatomical indications and cardiac mapping. Typically, a mapping catheter is chosen to desirably fit the pulmonary vein, adapting to the size and anatomical form of the pulmonary vein. The mapping catheter allows recording of the electrograms from the ostium of the pulmonary vein and from deep within the pulmonary vein; these electrograms are displayed and timed for the user. The treatment catheter 102 is initially placed deep within the pulmonary vein and gradually withdrawn to the ostium, proximal to the mapping catheter. Mapping and treatment then commences.

The current understanding of pulmonary vein electrophysiology is that most of the fibers in the pulmonary vein are circular and do not carry conduction into the vein. The electrical conduction pathways are longitudinal fibers which extend between the left atrium LA and the pulmonary vein. Pulmonary vein isolation is achieved by ablation of these connecting longitudinal fibers. For the left-sided pulmonary veins, pacing of the distal coronary sinus tends to increase the separation of the atrial signal and the pulmonary vein potential making these more electrically visible. The signals from within the pulmonary vein are evaluated. Each individual signal consists of a far field atrial signal, which is generally of low amplitude, and a sharp local pulmonary vein spike. The earliest pulmonary vein spike represents the site of the connection of the pulmonary vein and atrium. If the pulmonary vein spike and the atrial potential are examined, on some of the poles of the mapping catheter, these electrograms are widely separated, at other sites there will be a fusion potential of the atrial and PV signal. The latter indicate the sites of the longitudinal fibers and the potential sites for treatment.

In some embodiments, the tissue surrounding the opening of the left inferior pulmonary vein LIPV is treated in a point by point fashion with the use of the treatment catheter 102 (with assistance of mapping) to create a circular treatment zone around the left inferior pulmonary vein LIPV, as illustrated in FIG. 3. In some instances, specialized navigation software can be used to allow appropriate positioning of the treatment catheter 120. The delivery electrode 122 is positioned near or against the target tissue area, and energy is provided to the delivery electrode 122 so as to create a treatment area A. Since the energy is delivered to a localized area (focal delivery), the electrical energy is concentrated over a smaller surface area, resulting in stronger effects than delivery through an electrode extending circumferentially around the lumen or ostium. It also forces the electrical energy to be delivered in a staged regional approach, mitigating the potential effect of preferential current pathways through the surrounding tissue. These preferential current pathways are regions with electrical characteristics that induce locally increased electric current flow therethrough rather than through adjacent regions. Such pathways can result in an irregular electric current distribution around the circumference of a targeted lumen, which thus can distort the electric field and cause an irregular increase in treatment effect for some regions and a lower treatment effect in other regions. This may be mitigated or avoided with the use of focal therapy which stabilizes the treatment effect around the circumference of the targeted region. Thus, by providing the energy to certain regions at a time, the electrical energy is “forced” across different regions of the circumference, ensuring an improved degree of treatment circumferential regularity. FIG. 4 illustrates the repeated application of energy in point by point fashion around the left inferior pulmonary vein LIPV with the use of the treatment catheter 102 to create a circular treatment zone. As illustrated, in this embodiment each treatment area A overlaps an adjacent treatment area A so as to create a continuous treatment zone. The size and depth of each treatment area A may depend on a variety of factors, such as parameter values, treatment times, tissue characteristics, etc. It may be appreciated that the number of treatment areas A may vary depending on a variety of factors, particularly the unique conditions of each patient's anatomy and electrophysiology. In some embodiments, the number of treatment areas A include one, two, three, four, five, six, seven, eight, nine, ten, fifteen, twenty, twenty five, thirty or more.

When all the electrical connections between the atrium and the vein have been treated, there is electrical silence within the pulmonary vein, with only the far field atrial signal being recorded. Occasionally spikes of electrical activity are seen within the pulmonary vein with no conduction to the rest of the atrium; these clearly demonstrate electrical discontinuity of the vein from the rest of the atrial myocardium.

Additional treatment areas can be created at other locations to treat arrhythmias in either the right or left atrium dependent on the clinical presentation. Testing is then performed to ensure that each targeted pulmonary vein is effectively isolated from the body of the left atrium.

### Energy Delivery Algorithms

It may be appreciated that a variety of energy delivery algorithms 152 may be used. In some embodiments, the algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

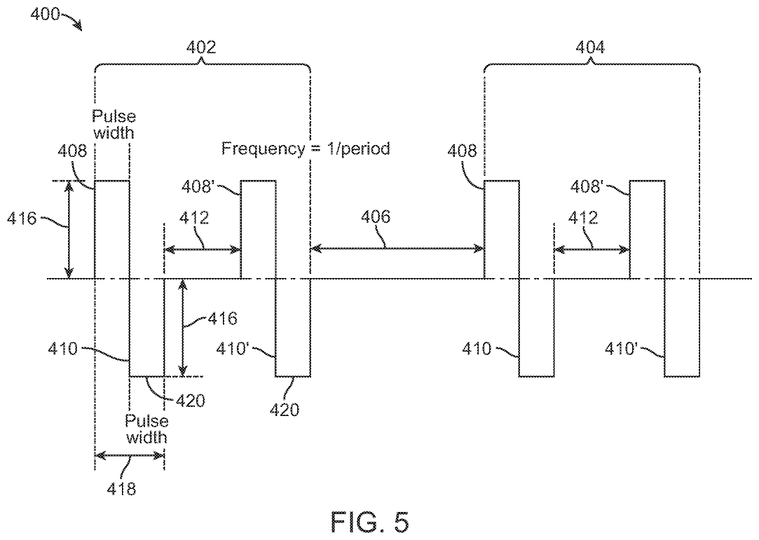

FIG. 5 illustrates an embodiment of a waveform 400 of a signal prescribed by an energy delivery algorithm 152. Here, two packets are shown, a first packet 402 and a second packet 404, wherein the packets 402, 404 are separated by a rest period 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time 412 (i.e. a pause) between each biphasic cycle. In this embodiment, the biphasic pulses are symmetric so that the set voltage 416 is the same for the positive and negative peaks. Here, the biphasic, symmetric waves are also square waves such that the magnitude and time of the positive voltage wave is approximately equal to the magnitude and time of the negative voltage wave.

### A. Voltage

The voltages used and considered may be the tops of square-waveforms, may be the peaks in sinusoidal or sawtooth waveforms, or may be the RMS voltage of sinusoidal or sawtooth waveforms. In some embodiments, the energy is delivered in a monopolar fashion and each high voltage pulse or the set voltage 416 is between about 500V to 10,000V, particularly about 1000V-2000V, 2000V-3000V, 3000V-3500V, 3500V-4000V, 3500V-5000V, 3500V-6000V, including all values and subranges in between including about 1000V, 2000V, 2500V, 2800V, 3000V, 3300V, 3500V, 3700V, 4000V, 4500V, 5000V, 5500V, 6000V to name a few.

It may be appreciated that the set voltage 416 may vary depending on whether the energy is delivered in a monopolar or bipolar fashion. In bipolar delivery, a lower voltage may be used due to the smaller, more directed electric field. The bipolar voltage selected for use in therapy is dependent on the separation distance of the electrodes, whereas the monopolar electrode configurations that use one or more distant dispersive pad electrodes may be delivered with less consideration for exact placement of the catheter electrode and dispersive electrode placed on the body. In monopolar electrode embodiments, larger voltages are typically used due to the dispersive behavior of the delivered energy through the body to reach the dispersive electrode, on the order of 10 cm to 100 cm effective separation distance. Conversely, in bipolar electrode configurations, the relatively close active regions of the electrodes, on the order of to 10 cm, including 1 mm to 1 cm, results in a greater influence on electrical energy concentration and effective dose delivered to the tissue from the separation distance. For instance, if the targeted voltage-to-distance ratio is 3000 V/cm to evoke the desired clinical effect at the appropriate tissue depth (1.3 mm), if the separation distance is changed from lmm to 1.2 mm, this would result in a necessary increase in treatment voltage from 300 to about 360 V, a change of 20%.

### B. Frequency

It may be appreciated that the number of biphasic cycles per second of time is the frequency when a signal is continuous. In some embodiments, biphasic pulses are utilized to reduce undesired muscle stimulation, particularly cardiac muscle stimulation. In other embodiments, the pulse waveform is monophasic and there is no clear inherent frequency. Instead, a fundamental frequency may be considered by doubling the monophasic pulse length to derive the frequency. In some embodiments, the signal has a frequency in the range 50 kHz-1 MHz, more particularly 50 kHz-1000 kHz. It may be appreciated that at some voltages, frequencies at or below 100-250 kHz may cause undesired muscle stimulation. Therefore, in some embodiments, the signal has a frequency in the range of 300-800 kHz, 400-800 kHz or 500-800 kHz, such as 300 kHz, 400 kHz, 450 kHz, 500 kHz, 550 kHz, 600 kHz, 650 kHz, 700 kHz, 750 kHz, 800 kHz. In addition, cardiac synchronization is typically utilized to reduce or avoid undesired cardiac muscle stimulation during sensitive rhythm periods. It may be appreciated that even higher frequencies may be used with components which minimize signal artifacts.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 600 kHz, while in other cases the treatment may be delivered using 2000 V with a waveform frequency of 400 kHz.

### D. Packets

As mentioned, the algorithm 152 typically prescribes a signal having a waveform comprising a series of energy packets wherein each energy packet comprises a series of high voltage pulses. The cycle count 420 is half the number of pulses within each biphasic packet. Referring to FIG. 5, the first packet 402 has a cycle count 420 of two (i.e. four biphasic pulses). In some embodiments, the cycle count 420 is set between 2 and 1000 per packet, including all values and subranges in between. In some embodiments, the cycle count 420 is 5-1000 per packet, 2-10 per packet, 2-20 per packet, 2-25 per packet, 10-20 per packet, 20 per packet, 20-30 per packet, 25 per packet, 20-40 per packet, 30 per packet, 20-50 per packet, 30-60 per packet, up to 60 per packet, up to 80 per packet, up to 100 per packet, up to 1,000 per packet or up to 2,000 per packet, including all values and subranges in between.

The packet duration is determined by the cycle count, among other factors. For a matching pulse duration (or sequence of positive and negative pulse durations for biphasic waveforms), the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

The number of packets delivered during treatment, or packet count, typically includes 1 to 250 packets including all values and subranges in between. In some embodiments, the number of packets delivered during treatment comprises 10 packets, 15 packets, 20 packets, 25 packets, 30 packets or greater than 30 packets.

### E. Rest Period

In some embodiments, the time between packets, referred to as the rest period 406, is set between about 0.001 seconds and about 5 seconds, including all values and subranges in between. In other embodiments, the rest period 406 ranges from about 0.01-0.1 seconds, including all values and subranges in between. In some embodiments, the rest period 406 is approximately 0.5 ms-500 ms, 1-250 ms, or 10-100 ms to name a few.

### F. Batches

In some embodiments, the signal is synced with the cardiac rhythm so that each packet is delivered synchronously within a designated period relative to the heartbeats, thus the rest periods coincide with the heartbeats. It may be appreciated that the packets that are delivered within each designated period relative to the heartbeats may be considered a batch or bundle. Thus, each batch has a desired number of packets so that at the end of a treatment period, the total desired number of packets have been delivered. Each batch may have the same number of packets, however in some embodiments, batches have varying numbers of packets.

In some embodiments, only one packet is delivered between heartbeats. In such instances, the rest period may be considered the same as the period between batches. However, when more than one packet is delivered between batches, the rest time is typically different than the period between batches. In such instances, the rest time is typically much smaller than the period between batches. In some embodiments, each batch includes 1-10 packets, 1-5 packets, 1-4 packets, 1-3 packets, 2-3 packets, 2 packets, 3 packets, 4 packets 5 packets, 5-10 packets, to name a few. In some embodiments, each batch has a period of 0.5 ms-1 sec, 1 ms-1 sec, 10 ms-1 sec, 10 ms-100 ms, to name a few. In some embodiments, the period between batches is variable, depending on the heart rate of the patient. In some instances, the period between batches is 0.25-5 seconds.

Treatment of a tissue area ensues until a desired number of batches are delivered to the tissue area. In some embodiments, 2-50 batches are delivered per treatment, wherein a treatment is considered treatment of a particular tissue area. In other embodiments, treatments include 5-40 batches, 5-30 batches, 5-20 batches, 5-10 batches, 5 batches, 6 batches, 7 batches, 8 batches, 9 batches, 10 batches, 10-15 batches, etc.

### G. Switch Time and Dead Time

A switch time is a delay or period of no energy that is delivered between the positive and negative peaks of a biphasic pulse, as illustrated in FIG. 5. In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microsecond, including all values and subranges in between.

Delays may also be interjected between each biphasic cycle, referred as “dead-time”. Dead time occurs within a packet, but between biphasic pulses. This is in contrast to rest periods which occur between packets. In other embodiments, the dead time 412 is in a range of approximately 0 to 0.5 microseconds, 0 to 10 microseconds, 2 to 5 microseconds, 0 to 20 microseconds, about 0 to about 100 microseconds, or about 0 to about 100 milliseconds, including all values and subranges in between. In some embodiments, the dead time 412 is in the range of 0.2 to 0.3 microseconds. Dead time may also be used to define a period between separate, monophasic, pulses within a packet.

Delays, such as switch times and dead times, are introduced to a packet to reduce the effects of biphasic cancellation within the waveform. In some instances, the switch time and dead time are both increased together to strengthen the effect. In other instances, only switch time or only dead time are increased to induce this effect.

### H. Waveforms

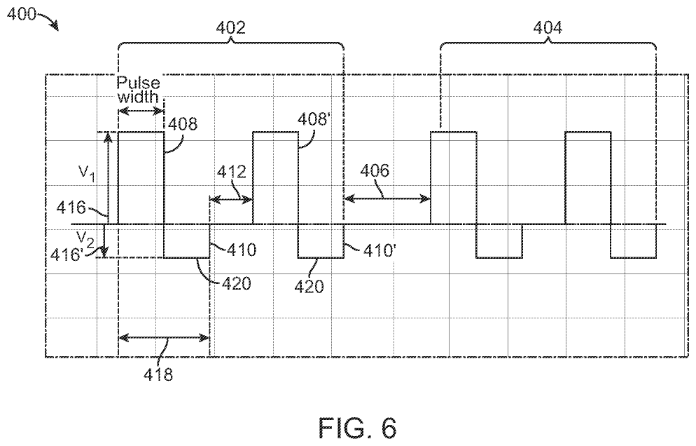

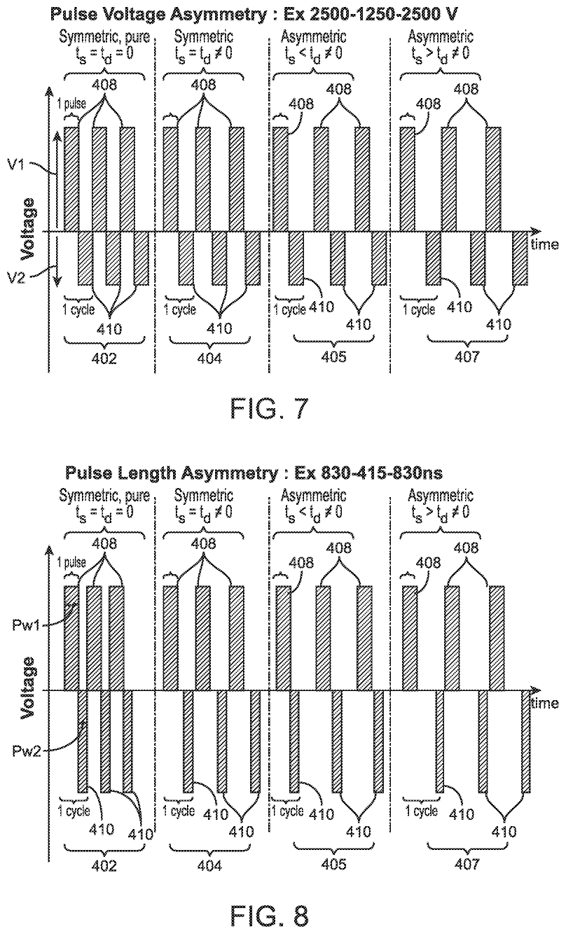

FIG. 5 illustrated an embodiment of a waveform 400 having symmetric pulses such that the voltage and duration of pulse in one direction (i.e., positive or negative) is equal to the voltage and duration of pulse in the other direction. FIG. 6 illustrates an example waveform 400 prescribed by another energy delivery algorithm 152 wherein the waveform 400 has voltage imbalance. Here, two packets are shown, a first packet 402 and a second packet 404, wherein the packets 402, 404 are separated by a rest period 406. In this embodiment, each packet 402, 404 is comprised of a first biphasic cycle (comprising a first positive pulse peak 408 having a first voltage V1 and a first negative pulse peak 410 having a second voltage V2) and a second biphasic cycle (comprising a second positive pulse peak 408′ having first voltage V1 and a second negative pulse peak 410′ having a second voltage V2). Here the first voltage V1 is greater than the second voltage V2. The first and second biphasic cycles are separated by dead time 412 between each pulse. Thus, the voltage in one direction (i.e., positive or negative) is greater than the voltage in the other direction so that the area under the positive portion of the curve does not equal the area under the negative portion of the curve. This unbalanced waveform may result in a more pronounced treatment effect as the dominant positive or negative amplitude leads to a longer duration of same charge cell membrane charge potential. In this embodiment, the first positive peak 408 has a set voltage 416 (V1) that is larger than the set voltage 416′ (V2) of the first negative peak 410. FIG. 7 illustrates further examples of waveforms having unequal voltages. Here, four different types of packets are shown in a single diagram for condensed illustration. The first packet 402 is comprised of pulses having unequal voltages but equal pulse widths, along with no switch times and dead times. Thus, the first packet 402 is comprised of four biphasic pulses, each comprising a positive peak 408 having a first voltage V1 and a negative peak 410 having a second voltage V2). Here the first voltage V1 is greater than the second voltage V2. The second packet 404 is comprised of pulses having unequal voltages but symmetric pulse widths (as in the first pulse 402), with switch times equal to dead times. The third packet 405 is comprised of pulses having unequal voltages but symmetric pulse widths (as in the first pulse 402), with switch times that are shorter than dead times. The fourth packet 407 is comprised of pulses having unequal voltages but symmetric pulse widths (as in the first pulse 402), with switch times that are greater than dead times. It may be appreciated that in some embodiments, the positive and negative phases of biphasic waveform are not identical, but are balanced, where the voltage in one direction (i.e., positive or negative), is greater than the voltage in the other direction but the length of the pulse is calculated such that the area under the curve of the positive phase equals the area under the curve of the negative phase.

In some embodiments, imbalance includes pulses having pulse widths of unequal duration. In some embodiments, the biphasic waveform is unbalanced, such that the voltage in one direction is equal to the voltage in the other direction, but the duration of one direction (i.e., positive or negative) is greater than the duration of the other direction, so that the area under the curve of the positive portion of the waveform does not equal the area under the negative portion of the waveform.

FIG. 8 illustrates further examples of waveforms having unequal pulse widths. Here, four different types of packets are shown in a single diagram for condensed illustration. The first packet 402 is comprised of pulses having equal voltages but unequal pulse widths, along with no switch times and dead times. Thus, the first packet 402 is comprised of four biphasic pulses, each comprising a positive peak 408 having a first pulse width PW1 and a negative peak 410 having a second pulse width PW2). Here the first pulse width PW1 is greater than the second pulse width PW2. The second packet 404 is comprised of pulses having equal voltages but unequal pulse widths (as in the first pulse 402), with switch times equal to dead times. The third packet 405 is comprised of pulses having equal voltages but unequal pulse widths (as in the first pulse 402), with switch times that are shorter than dead times. The fourth packet 407 is comprised of pulses having equal voltages but unequal pulse widths (as in the first pulse 402), with switch times that are greater than dead times.

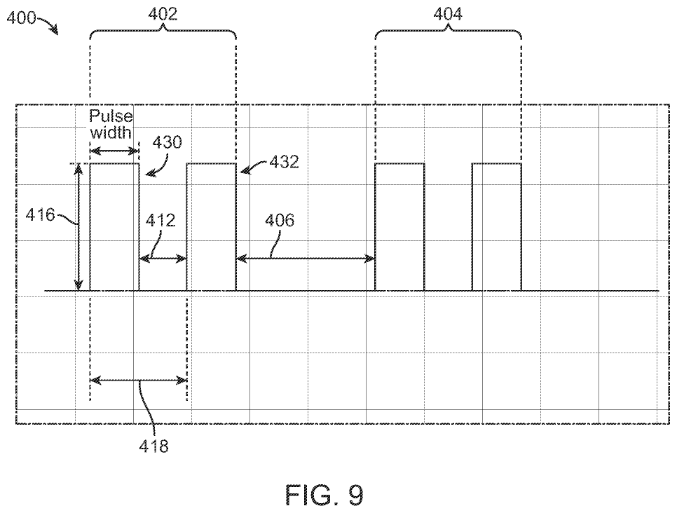

FIG. 9 illustrates an example waveform 400 prescribed by another energy delivery algorithm 152 wherein the waveform is monophasic, a special case of imbalance whereby there is only a positive or only a negative portion of the waveform. Here, two packets are shown, a first packet 402 and a second packet 404, wherein the packets 402, 404 are separated by a rest period 406. In this embodiment, each packet 402, 404 is comprised of a first monophasic pulse 430 and a second monophasic pulse 432. The first and second monophasic pulses 430, 432 are separated by dead time 412 between each pulse. This monophasic waveform could lead to a more desirable treatment effect as the same charge cell membrane potential is maintain for longer durations. However, adjacent muscle groups will be more stimulated by the monophasic waveform, compared to a biphasic waveform.

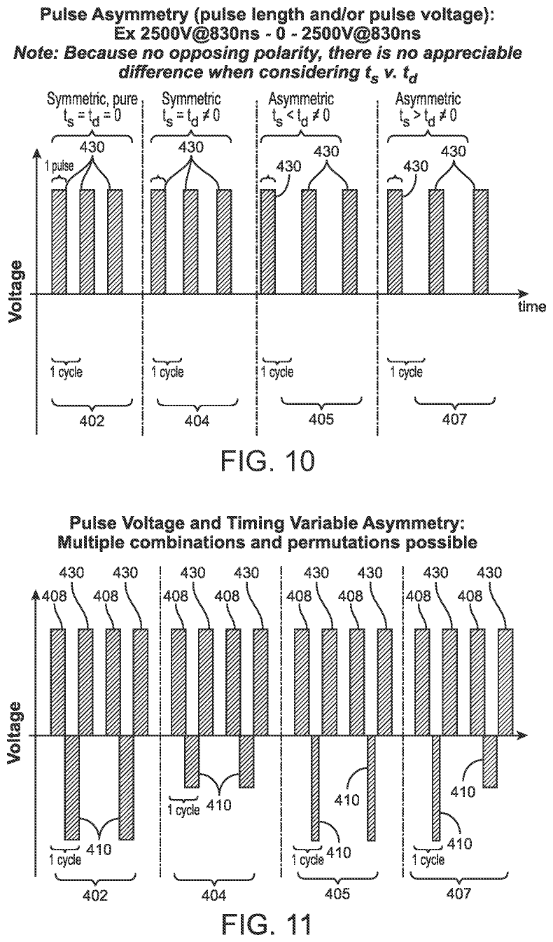

FIG. 10 illustrates further examples of waveforms having monophasic pulses. Here, four different types of packets are shown in a single diagram for condensed illustration. The first packet 402 is comprised of pulses having identical voltages and pulse widths, with no switch times (because the pulses are monophasic) and a dead time equal to the active time. In some cases, there may be less dead time duration than the active time of a given pulse. Thus, the first packet 402 is comprised of three monophasic pulses 430, each comprising a positive peak. In instances where the dead time is equal to the active time, the waveform may be considered unbalanced with a fundamental frequency representing a cycle period of 2× the active time and no dead time. The second packet 404 is comprised of monophasic pulses 430 having equal voltages and pulse widths (as in the first packet 402), with larger dead times. The third packet 405 is comprised of monophasic pulses 430 having equal voltages and pulse widths (as in the first packet 402), and even larger dead times. The fourth packet 407 is comprised of monophasic pulses 430 having equal voltages and pulse widths (as in the first packet 402), with yet larger dead times.

In some embodiments, an unbalanced waveform is achieved by delivering more than one pulse in one polarity before reversing to an unequal number of pulses in the opposite polarity. FIG. 11 illustrates further examples of waveforms having such phase imbalances. Here, four different types of packets are shown in a single diagram for condensed illustration. The first packet 402 is comprised of four cycles having equal voltages and pulse widths, however, opposite polarity pulses are intermixed with monophasic pulses. Thus, the first cycle comprises a positive peak 408 and a negative peak 410. The second cycle is monophasic, comprising a single positive pulse with no subsequent negative pulse 430. This then repeats. The second packet 404 is comprised of intermixed biphasic and monophasic cycles (as in the first packet 402), however the pulses have unequal voltages. The third packet 405 is comprised of intermixed biphasic and monophasic cycles (as in the first packet 402), however the pulses have unequal pulse widths. The fourth packet 407 is comprised of intermixed biphasic and monophasic pulses (as in the first packet 402), however the pulses have unequal voltages and unequal pulse widths. Thus, multiple combinations and permutations are possible.

### I. Waveform Shapes

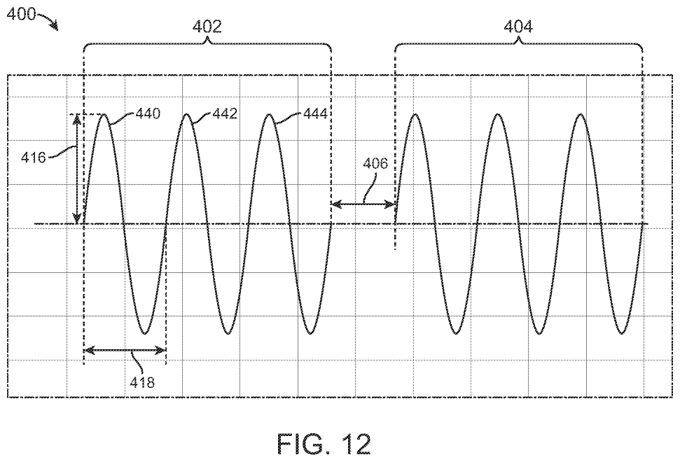

FIG. 12 illustrates an example waveform 400 prescribed by another energy delivery algorithm 152 wherein the pulses are sinusoidal in shape rather than square. Again, two packets are shown, a first packet 402 and a second packet 404, wherein the packets 402, 404 are separated by a rest period 406. In this embodiment, each packet 402, 404 is comprised three biphasic pulses 440, 442, 444. And, rather than square waves, these pulses 440, 442, 444 are sinusoidal in shape. One benefit of a sinusoidal shape is that it is balanced or symmetrical, whereby each phase is equal in shape. Balancing may assist in reducing undesired muscle stimulation. It may be appreciated that in other embodiments the pulses have decay-shaped waveforms.

Energy delivery may be actuated by a variety of mechanisms, such as with the use of a button 164 on the catheter 102 or a foot switch 168 operatively connected to the generator 104. Such actuation typically provides a single energy dose. The energy dose is defined by the number of packets delivered and the voltage of the packets. Each energy dose delivered to the tissue maintains the temperature at or in the tissue below a threshold for thermal ablation. In addition, the doses may be titrated or moderated over time so as to further reduce or eliminate thermal build up during the treatment procedure. Instead of inducing thermal damage, defined as protein coagulation at sites of danger to therapy, the energy dose provide energy at a level which induces treats the condition without damaging sensitive tissues.

### Treatment Catheter Designs

The systems and devices described herein may be used with a variety of types and styles of treatment catheters 102. In some embodiments, the treatment catheters 102 are designed to deliver focal therapy and in other embodiments, the treatment catheters 102 are designed to deliver “one shot” therapy. Focal therapy is considered to be a therapy wherein the energy is delivered in a sequence, such as the repeated application of energy in point by point fashion, such as around a pulmonary vein to create a circular treatment zone, such as previously illustrated in FIG. 4, or along a line, curve, etc. One shot therapy is considered to be a therapy wherein the energy is delivered via an energy delivery body or delivery electrode(s) to the entire circumference of the entrance to the pulmonary vein in “one shot”, however such delivery may be repeated if desired. This may optionally include rotation of the energy delivery body or electrode 122 between “shots” if desired.

### Focal Therapy

As mentioned previously, focal therapy is often performed with the use of a delivery electrode 122 having a cylindrical shape with a distal face, such as illustrated in FIGS. 2A-2B. Here the distal face is flat and circular. In some embodiments, the distal face of the delivery electrode 122 has a diameter of 2-3 mm. In such embodiments, when the treatment catheter 102 is positioned perpendicularly to the tissue, the distal face is able to cover a portion of tissue having a diameter of 2-3 mm. However, a larger portion of tissue may be covered with alternative device designs described herein. Such larger coverage may provide larger lesion sizes with a single application.

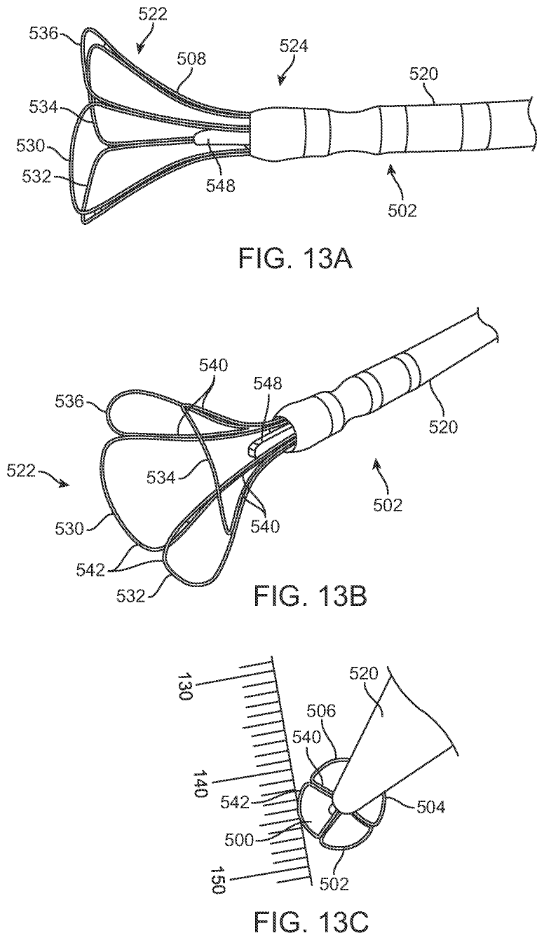

FIGS. 13A-13C illustrate an embodiment of a treatment catheter 502 configured for focal delivery that optionally covers a larger area of tissue than a cylindrically shaped delivery electrode typically found on conventional RF catheters. Here, the catheter 502 comprises an elongate shaft 520 having at an energy delivery body near its distal end 524, wherein the energy delivery body comprises at least one delivery electrode 522. In particular, in this embodiment, the catheter 502 includes a plurality of electrodes that are arranged to form a continuous shape (i.e. a continuous outer rim) and therefore, optionally, a continuous lesion. A continuous lesion can be formed by energizing the plurality of the electrodes, either simultaneously or non-simultaneously, however it may be appreciated that in some embodiments a subset (including just one) of the plurality of electrodes may be energized if desired so as to create a lesion that is less than the continuous shape. It may also be appreciated that in such instances a continuous lesion may be ultimately created by manipulation of the catheter 502, such as by rotation or repositioning, if it is not desired to create a continuous lesion in the initial position.

FIG. 13A provides a side view of the catheter 502, wherein the at least one delivery electrode 522 comprises four loop-shaped electrodes: a first electrode 530, a second electrode 532, a third electrode 534 and a fourth electrode 536. Each of the plurality of electrodes 530, 532, 534, 536 is comprised of a wire 508 that is formed or shaped into a petal or loop shape having a narrower shape near the shaft 520 and a larger, wider shape extending away from the shaft 520. Thus, the electrodes 530, 532, 534, 536 fan out or extend outwardly from the shaft 520 in a flower bloom shape, as shown in the perspective view of FIG. 13B. In this embodiment, the sides 540 of each of the loop shapes are disposed adjacent to each other and are joined together. Thus, the first electrode 530 is joined on one side to the adjacent second electrode 532 and on the opposite side to the adjacent fourth electrode 536. This provides stability to the overall design, maintaining the relative position of the electrodes 530, 532, 534, 536 throughout delivery and use. In some embodiments, the sides 540 are joined with a material that insulates the sides 540 so as to prevent conduction of energy therethrough. This concentrates energy delivery through the distal edges 542 of each of the loop shapes which is configured to contact the tissue.

FIG. 13C provides a top-down view of the electrodes 530, 532, 534, 536 pressed against a surface. As shown, the distal edges 542 of each loop shape contact the surface and together form a ring or circular shape, wherein the side 540 appear as “spokes” to the “wheel”. When the distal edges 542 are pressed against tissue and energy is delivered therethrough. Typically, the resulting lesion is larger than the width of the wire 508. Therefore, any small gaps between the loop shapes will not diminish the lesion since the resulting lesion overwhelms any effect of small gaps. In some embodiments, the wire 508 has a diameter of 0.0075 inches and the resulting lesion has a width of 12 mm (i.e. measured across the width of the wire). Thus, in some embodiments, the lesion has a ring shape, circular shape or donut shape. It may be appreciated that in some embodiments, the lesion is large enough to connect through the center of the ring shape so that the lesion appears to be a solid circle. In some embodiments, the catheter 502 includes an additional central electrode 548, such as illustrated in FIGS. 13A-13C. The central electrode 548 extends distally from the central or longitudinal axis of the shaft 520. In some embodiments, the central electrode 548 does not extend longitudinally to the plane of the distal edges 542 when in the relaxed position, as illustrated in FIGS. 13A-13B. In such embodiments, the central electrode 548 contacts the surface of the tissue when the distal edges 542 are pressed against the surface of the tissue, splaying the loops apart so that they flatten against the tissue as in FIG. 13C. Delivery of energy through the central electrode 548 in this position creates a central lesion which assists in creating a continuous lesion within the footprint of the loop shaped electrodes 530, 532, 534, 536.

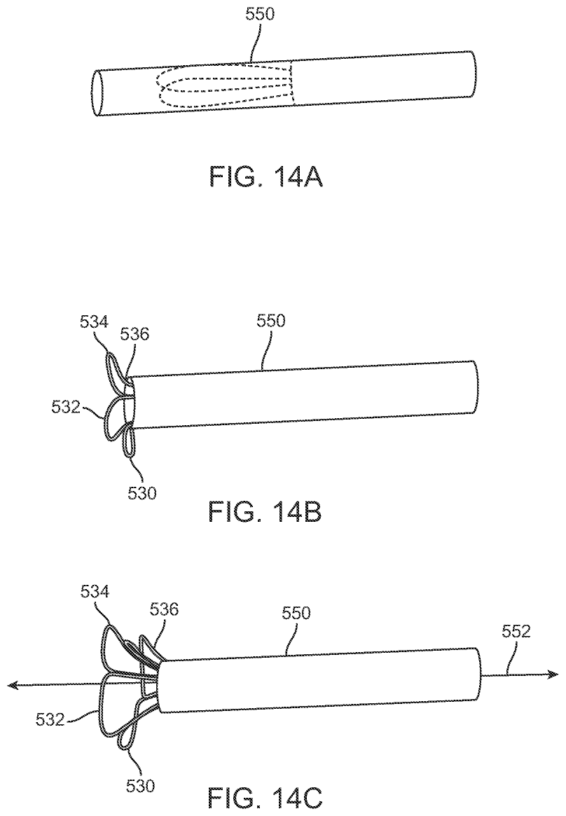

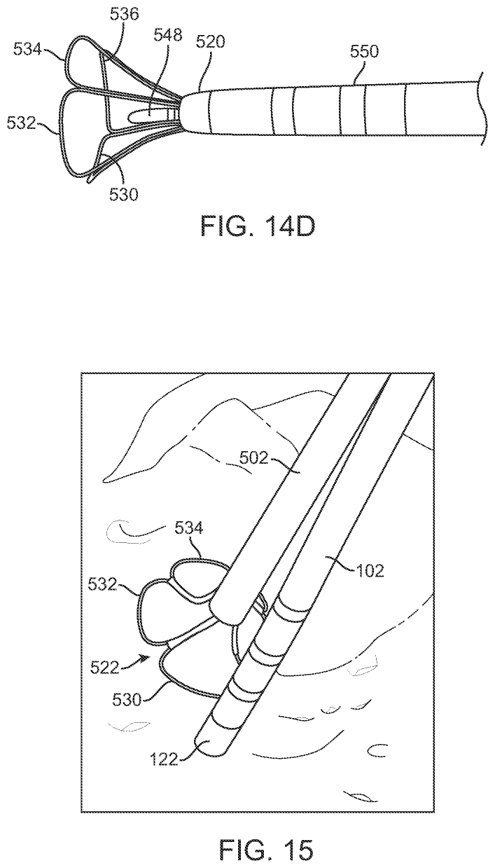

FIGS. 14A-14D illustrate delivery of the embodiment of the catheter 502 of FIGS. 13A-13C. It may be appreciated that the wire 508 may be comprised of a variety of materials, such as nitinol or drawn filled tube (e.g. 10% platinum/nitinol), to name a few. In this embodiment, the wire 508 is flexible so as to collapse within a delivery sheath 550, as illustrated in FIG. 14A. This allows the distal end of the catheter 502 to be successfully advanced to the target tissue site. Typically, the delivery sheath 550 has an inner diameter in the range of 2.5 mm to 3.5 mm. Incremental exposure of the electrodes 530, 532, 534, 536 can be achieved by advancement of the catheter 502 within the delivery sheath 550 or the delivery sheath 550 may be retracted to reveal the distal end of the catheter 502. FIG. 14B illustrates the distal end of the catheter 502 emerging from the delivery sheath 550, wherein the loop shaped electrodes 530, 532, 534, 536 begin splaying outwardly. FIG. 14C illustrates the catheter 502 further emerged from the delivery sheath 550, wherein the loop shaped electrodes 530, 532, 534, 536 expand further radially outward from the longitudinal axis 552 of both the catheter 502 and the delivery sheath 550. FIG. 14D illustrates full exposure of the loop shaped electrodes 530, 532, 534, 536 wherein the loop shaped electrodes 530, 532, 534, 536 are able to recoil to their fullest relaxed expansion. It may be appreciated that pressing the loop shaped electrodes 530, 532, 534, 536 against a surface may further expand the loop shaped electrodes 530, 532, 534, 536 to create an even larger footprint. In some embodiments, the largest diameter is 9-15 mm which may create a lesion with a similar or larger diameter due to electric field effects.

In some embodiments, the overall lesion size created by the footprint of the treatment catheter 502 of FIGS. 13A-13C is larger than the lesion size created by the footprint of the solid tipped treatment catheter 102 of FIGS. 2A-2B. FIG. 15 provides a visual illustration of the respective end effectors adjacent to each other in contact with tissue. Here, the solid tipped treatment catheter 102 has a delivery electrode 122 having a circular face of 3 mm in diameter. The treatment catheter 502 has a delivery electrode 522 comprises four loop-shaped electrodes wherein together the electrodes 530, 532, 534, 536 form a circular face of 9-10 mm in diameter. This larger footprint is at least three times the size of the smaller footprint. In some instances, the larger footprint is up to six times the size of the smaller footprint. Thus, the difference in size between the delivery electrodes and therefore footprints is typically in the range of 3-6 times. Since the delivery electrode 122 of the solid tipped treatment catheter 102 typically has the same diameter as its shaft, the size of the delivery electrode 522 in its expanded configuration has the same relationship to the shaft of the treatment catheter 502 as it does to the solid tipped treatment catheter 102 (i.e. the delivery electrode in its expanded configuration is 3-6 times the diameter of the shaft of the treatment catheter 502). This larger diameter footprint can provide a number of advantages. In some instances, the larger footprint allows the user to perform a complete treatment protocol with less lesions. For example, when circling a pulmonary vein with the use of a solid tipped treatment catheter 102, as illustrated in FIG. 4, the user may create 35 lesions to complete the full circle lesion. However, when performing this same procedure with the treatment catheter 502 of FIGS. 13A-13C, the user may create the full circle lesion with 12 lesions. It may be appreciated that the treatment catheter 502 may be configured to have footprints of a variety of different diameters, and such diameters will proportionally correspond to the number of lesions desired to create a complete circle. Such logic follows other shaped lesions, such as line lesions and other types of treatments. Often times, the larger the lesion, the lesser the number of lesions is utilized to perform a treatment. This typically reduces the treatment time and leads to shorter procedures. Another advantage of the loop-shaped electrode design is the ability to create a larger footprint while maintaining healthy tissue within the center of the footprint. Thus, when ring shaped lesions are created surrounding healthy tissue, more of the healthy cardiac tissue is maintained than if the lesion were a solid disk shape. Adjacent ring-shaped lesions are able to block conduction through the cardiac tissue as effectively as solid disk-shaped lesions while maintaining a higher percentage of healthy tissue. In some embodiments, the ring shape maintains 25% more healthy tissue.

It may be appreciated that, in some embodiments, the overall diameter or footprint size may be controlled by adjusting the deployment of the loop shaped electrodes 530, 532, 534, 536 from the delivery sheath 550. For example, a smaller diameter may be achieved by only advancing the loop shaped electrodes 530, 532, 534, 536 partially from the sheath 550, such as in FIGS. 14B-14C. Likewise, a larger diameter may be achieved by full advancement of the loop shaped electrodes 530, 532, 534, 536 from the sheath 550. In addition, a variety of catheters 502 may be designed having energy delivery electrodes 522 of differing maximum diameter to suit a variety of needs.

It may also be appreciated that the overall diameter or footprint size may be configured so that the delivery electrode 522 is able to provide one-shot therapy. Again, one shot therapy is considered to be a therapy wherein the energy is delivered via the delivery electrode 522 to the entire treatment area, such as the circumference of the entrance to the pulmonary vein, in “one shot”, however such delivery may be repeated if desired. This may optionally include rotation of the electrode 122 between “shots” if desired. In such embodiments, the overall diameter may be in the range of 22 to 33 mm.

It may be appreciated that the energy delivery body or energy delivery electrode 522 of the treatment catheter 502 may have any suitable number of loop-shaped electrodes, including two, three, four, five, six, seven, eight, nine, ten or more. Likewise, the electrodes may be energizable together or independently. When the electrodes are energized independently, the electrodes may be energized sequentially or in various patterns. Likewise, in some embodiments a subset (including just one) of the plurality of electrodes may be energized. This provides a wide variety of options to create desired lesions.

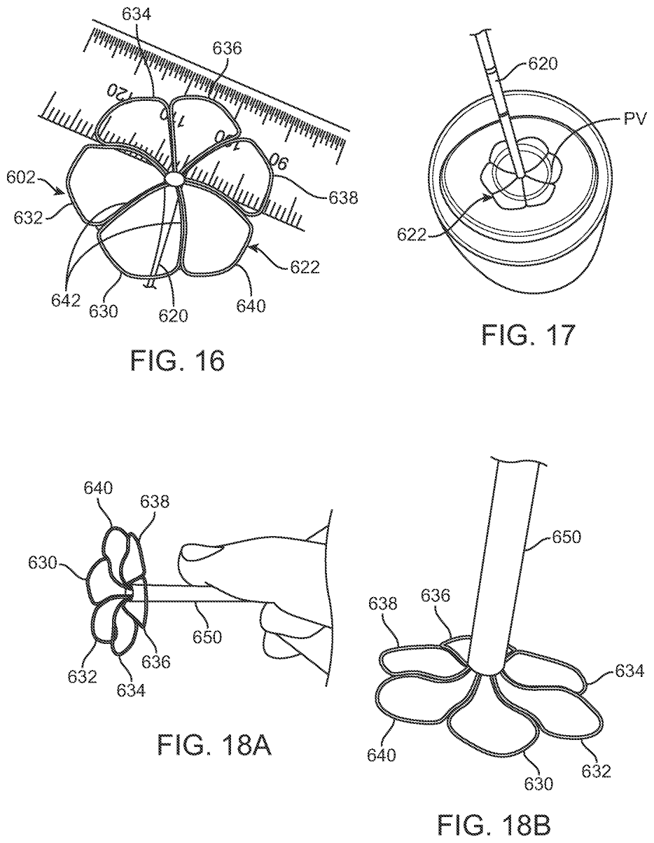

FIG. 16 illustrates another embodiment of a treatment catheter 602 configured for focal delivery, that optionally covers a larger area of tissue than a cylindrically shaped delivery electrode typically found on conventional RF catheters, or for one shot therapy. This embodiment is similar to the embodiment of FIGS. 13A-13C in that the catheter 602 includes a plurality of loop-shaped electrodes that are arranged to form a continuous shape (i.e. a continuous outer rim) and therefore, optionally, a continuous lesion. In this embodiment, the delivery electrode 622 comprises six loop-shaped electrodes (rather than four): a first electrode 630, a second electrode 632, a third electrode 634, a fourth electrode 636, a fifth electrode 638 and a sixth electrode 640. However, it may be appreciated that any number of electrodes may be utilized, such as up to 10-12 electrodes. Again, a continuous lesion can be formed by energizing the plurality of the electrodes, either simultaneously or non-simultaneously, however it may be appreciated that in some embodiments a subset (including just one) of the plurality of electrodes may be energized if desired so as to create a lesion that is less than the continuous shape. It may also be appreciated that in such instances a continuous lesion may be ultimately created by manipulation of the catheter 602, such as by rotation or repositioning, if it is not desired to create a continuous lesion in the initial position.

As shown in FIG. 16, each of the plurality of electrodes 630, 632, 634, 636, 638, 640 fan out or extend outwardly from the shaft 620 in a flower bloom shape upon actuation. In this embodiment, the sides 642 of each of the loop shapes are disposed adjacent to each other and are joined together. Thus, the first electrode 630 is joined on one side to the adjacent second electrode 632 and on the opposite side to the adjacent sixth electrode 640.

It may be appreciated that the overall diameter of the energy delivery body or delivery electrode 622 may be configured for focal therapy, for one shot therapy or for both. FIG. 16 illustrates an embodiment sized for one shot therapy wherein the overall diameter or footprint size is 30 mm, as indicated by the ruler measurement. It may be appreciated that such diameters are typically in the range of 22 to 33 mm for one shot therapy. Likewise, FIG. 17 illustrates the embodiment of FIG. 16 positioned against a laboratory benchtop model of the entrance to the pulmonary vein PV. As shown, together the outer rims of the electrodes 630, 632, 634, 636, 638, 640 extend around the circumference of the model entrance to the pulmonary vein so as to provide one shot therapy. It may be appreciated that the dimensions may be adjusted for focal delivery, either by progressive deployment (similar to FIGS. 14A-14D) or by generation of a delivery electrode 622 having a smaller overall diameter, such as is in the range of 9-15 mm. Combination use for focal and one shot therapy will be described herein.

FIGS. 18A-18B illustrate the delivery electrode 622 deployed from a delivery sheath 650 so that the electrodes 630, 632, 634, 636, 638, 640 extend substantially perpendicular to a longitudinal axis of the delivery sheath 650. In this configuration, the external rims of the electrodes 630, 632, 634, 636, 638, 640 have expanded to reach their maximum diameter or footprint size. FIG. 18B illustrates the delivery electrode 622 positioned against a flat surface, such as representing a tissue plane, which illustrates that the electrodes 630, 632, 634, 636, 638, 640 are able to lay substantially flat against the surface.

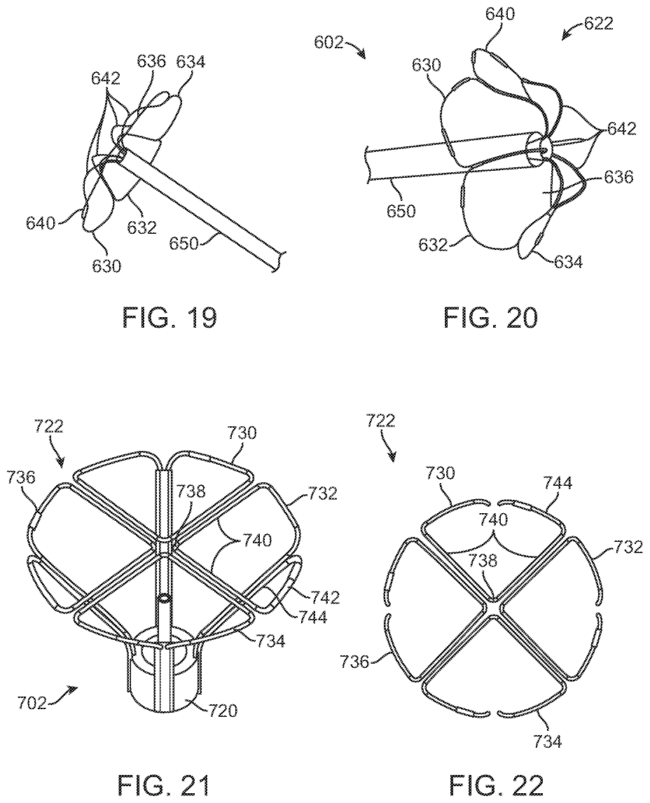

Further extension of the electrodes 630, 632, 634, 636, 638, 640 from the sheath 650 allows the petal shaped electrodes 630, 632, 634, 636, 638, 640 to curve downward so that the external rims of the electrodes 630, 632, 634, 636, 638, 640 are disposed proximally of the distal tip of the sheath 650, as illustrated in FIG. 19. This is achieved by pre-formed curvatures of the sides 640 which are allowed to recoil toward their pre-formed shape upon further release. The pre-curvature causes the sides 640 to arc distally and then bend proximally so that the overall shape of the delivery electrode 622 resembles an umbrella or a mushroom cap. In this configuration, the delivery electrode 622 is preferentially arranged to deliver energy to surfaces within a lumen, such as within a pulmonary vein PV. Here, positioning of the delivery electrode 622 against an entrance of a pulmonary vein PV allows the external rim of the electrodes 630, 632, 634, 636, 638, 640 to reside along the circumference of the pulmonary vein PV while the sides 642 extend into the pulmonary vein PV, along the inner lumen of the pulmonary vein PV. In some embodiments, the sides 642 are insulated so that such positioning provides stability while energy is delivered via the outer rim of the electrodes 630, 632, 634, 636, 638, 640. In other embodiments, the sides 642 are not insulated so that such positioning allows delivery of energy to portions of the inner lumen of the pulmonary vein PV via the sides 642. This may assist in creating larger or more complex lesions.

Further deployment of the electrodes 630, 632, 634, 636, 638, 640 exaggerates this shape wherein the sides 640 are allowed to bend or arc even further, as illustrated in FIG. 20. In this configuration, the delivery electrode 622 is preferentially arranged to deliver focal energy to a surface of tissue by positioning the sides 642 against the surface. In such an arrangement, the delivery electrode 622 may be used so that the outer rims of the electrodes 630, 632, 634, 636, 638, 640 are not in contact with the tissue and the energy is delivered to the tissue via the sides 642. Due to the rounded curvature of the sides 642. the delivery electrode 622 is able to be “rolled” along the tissue, such as having a ball shape wherein the curved surfaces are able to engage the tissue by tilting the shaft 620 (within the sheath 650) of the catheter 602. This provides unlimited engagement positions and high flexibility in energy delivery. Thus, this embodiment can transition between configurations to provide either one shot therapy or focal therapy; therefore it is particularly suited for combination use.

It may be appreciated that the embodiment of FIGS. 16-20 can optionally deliver via the external rims of the electrodes 630, 632, 634, 636, 638, 640, through the sides 642 of the pedal shapes or through both, either simultaneously, alternatively or in any combination. Thus, a ring or donut shaped lesion can be created or a solid circular shape lesion can be created, each of various sizes.

FIG. 21 illustrates an embodiment of a treatment catheter 702 configured for focal delivery rather than one shot delivery or combination delivery. Here, the lesions formed have a solid circular shape and are therefore primarily suitable for treating tissue surfaces, such as in a point-by-point fashion. This is due to the shape and configuration of its delivery electrode 722. FIG. 22 illustrates an embodiment of the treatment catheter 702 wherein the energy delivery body or delivery electrode 722 comprises a plurality of trowel shaped electrodes 730, 732, 734, 736 extending from a shaft 720. Each trowel shaped electrode has a substantially triangular shape wherein a tip 738 of the triangular shape resides near the center of the formed lesion, the sides 740 of the triangular shape extend radially outwardly from the center of the formed lesion and the base 742 of the triangular shape extends along the periphery of the formed lesion. In this embodiment, the tip 738 is a free end and the base 742 is attached to the catheter 702 by a support 744. Thus, the tips 738 of the trowel shaped electrodes 730, 732, 734, 736 are able to flex distally and proximally to accommodate structural variations in the surface of the tissue against which the delivery electrode 722 is placed. The tips 738 and sides 740 of the trowel shaped electrodes 730, 732, 734, 736 also help generate a continuous lesion, rather than a donut shaped lesion.

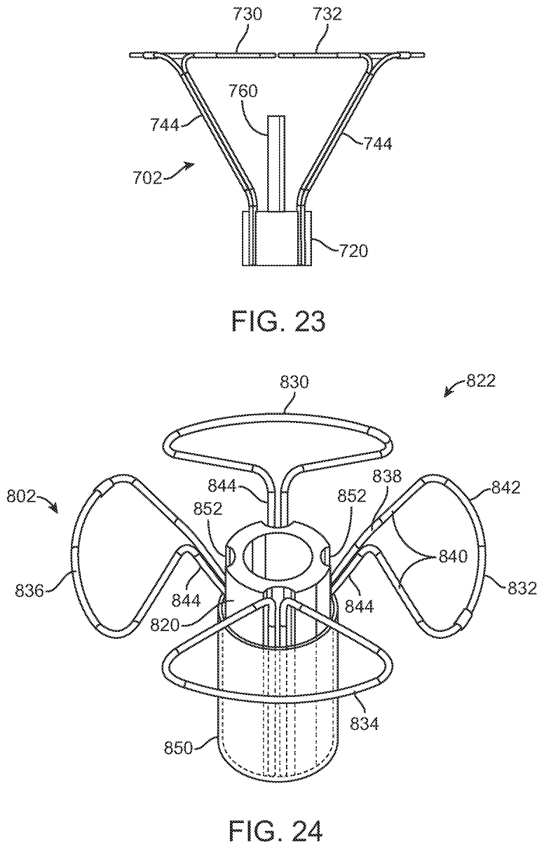

FIG. 23 illustrates a side view of an embodiment of a treatment catheter 702 similar to that of FIGS. 21-22. Here, only two trowel shaped electrodes 730, 732 are visible. As shown, the trowel shaped electrodes 730, 732 align along a plane perpendicular the shaft 720. The plane is spaced distally from the distal end of the shaft 720 determined by the length of the supports 744. In some embodiments, irrigation is provided by an irrigation lumen 760 such as extending from the distal end of the shaft 720. This allows delivery of irrigation fluid in the area of lesion formation.

It may be appreciated that any suitable number of trowel shaped electrodes may be present, typically three, four, five, six, seven, eight or more. It may also be appreciated that the trowel shaped electrodes may be independently activated, activated together or activated in any combination, such as in pairs, groups or in a sequential pattern of individual or grouped electrodes.

It may also be appreciated that any portion of the trowel shaped electrodes may be insulated to focus energy delivery through a particular area. In this embodiment, the supports 744 are insulated so as to direct energy through the triangular shaped portions of the trowel shaped electrodes. It may also be appreciated that various sensors, such as microsensors for contact feedback or electroanatomic mapping systems, may be positioned along the trowel shaped electrodes. In this embodiment, such sensors are disposed along the bases 744, however sensors may be positioned along any suitable portion.

FIG. 24 illustrates another embodiment of a treatment catheter 802. Here the delivery electrode 822 comprises a plurality of trowel shaped electrodes 830, 832, 834, 836 extending from a shaft 820. Again, each trowel shaped electrode has a substantially triangular shape wherein a tip 838 of the triangular shape resides near the center of the formed lesion, the sides 840 of the triangular shape extend radially outwardly from the center of the formed lesion and the base 842 of the triangular shape extends along the periphery of the formed lesion. However, in this embodiment, the base 842 is a free end and the tip 838 is attached to the catheter 802 by a support 844. In some embodiments, the supports 844 are pre-curved so as to bias the radially outwardly from the shaft 820. Typically, the supports 844 are comprised of material that provide flexibility and springiness so as to allow the supports 844 to bend toward the shaft 820 and then recoil upon release to the pre-curved configuration. This provides the ability to move the trowel shaped electrodes 830, 832, 834, 836, such as to create different sized lesions. In this embodiment, the supports 844 extend along at least a portion of the shaft 820, such as within longitudinal grooves 852 along the shaft 820. Likewise, in this embodiment a sheath 850 is advanceable over the shaft 820 and the grooves 852, holding the supports 844 within the grooves 852. Upon retraction of the sheath 850, the supports 844 are able to recoil toward their pre-curved configuration, moving the trowel shaped electrodes 830, 832, 834, 836 radially outwardly as shown. It may be appreciated that the amount of movement may be determined by the amount of retraction of the sheath 850. Maximum retraction allows maximum expansion of the trowel shaped electrodes 830, 832, 834, 836 so as to create a maximum sized lesion. Smaller lesions may be created by incrementally advancing the sheath 850 so as to desirably position the trowel shaped electrodes 830, 832, 834, 836.

It may be appreciated that any suitable number of trowel shaped electrodes may be present, typically three, four, five, six, seven, eight or more. It may also be appreciated that the trowel shaped electrodes may be independently activated, activated together or activated in any combination, such as in pairs, groups or in a sequential pattern of individual or grouped electrodes.

It may also be appreciated that any portion of the trowel shaped electrodes may be insulated to focus energy delivery through a particular area. In this embodiment, the supports 844 are insulated so as to direct energy through the triangular shaped portions of the trowel shaped electrodes. It may also be appreciated that various sensors, such as microsensors for contact feedback or electroanatomic mapping systems, may be positioned along the trowel shaped electrodes. In this embodiment, such sensors are disposed along the bases 844, however sensors may be positioned along any suitable portion.

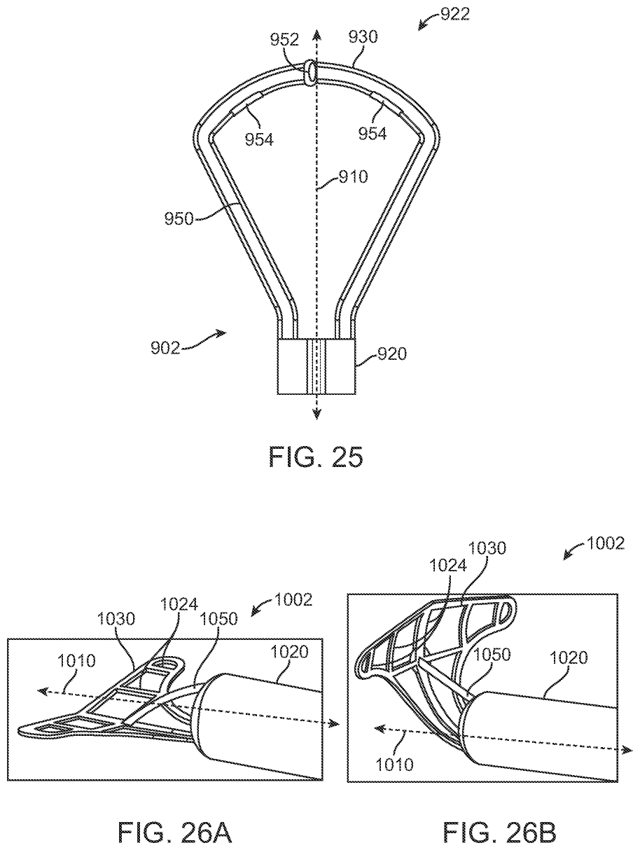

FIG. 25 illustrates another embodiment of a treatment catheter 902. Here the delivery electrode 922 comprises a single petal, paddle or loop shaped electrode 930 having a narrower shape near the shaft 920 and a larger, wider shape extending away from the shaft 920. In this embodiment, the electrode 930 resides in a plane aligned with a longitudinal axis 910 of the shaft 920. However, in this embodiment, the electrode 930 is comprised of a flexible material which allows the electrode 930 to bend into a variety of planes relative to the longitudinal axis 910 including perpendicular to the longitudinal axis 910. Bending of the electrode 930 allows the electrode 930 to be positioned against a variety of tissue surfaces from a variety of different approaches. For example, the electrode 930 is not limited to approaching a target tissue from a substantially perpendicular approach but can approach a target tissue from a parallel approach. Thus, the catheter 902 may be advanced along tissue in a plane parallel to the tissue until the electrode 930 is positioned adjacent the target tissue. The shaft 920 may then be angled away from the tissue so that the electrode 930 engages the tissue. The shaft 920 may then be further angled away from the tissue to increase engagement and/or contact force of the electrode 930 with the target tissue. This may be particularly useful when approaching tissue within a lumen, such as a pulmonary vein.

In this embodiment, the catheter 902 further comprises a sensing loop 950 that is similar in shape to the single petal, paddle or loop shaped electrode 930 but is smaller so as to reside inside the loop shape of the electrode 930, as illustrated in FIG. 25. In this embodiment, the sensing loop 950 has similar flexibility to the electrode 930 so it can act in symmetry with the electrode 930. Thus, in this embodiment the sensing loop 950 and the electrode 930 are connected by a joiner 952 to ensure that they remain substantially in the same plane. The sensing loop 950 is comprised of one or more sensors 954, such as microsensors for contact feedback or electroanatomic mapping systems.

FIGS. 26A-26B illustrate another embodiment of a treatment catheter 1002 having a paddle shaped delivery electrode 1022. Here the delivery electrode 1022 comprises a single hammerhead paddle shaped electrode 1030 having a narrower shape near the shaft 1020 and a wider, hammerhead shape distal from the shaft 1020. In addition, the electrode 1030 includes a plurality of crossbeams 1024 internal to the overall hammerhead shape which provide support for the hammerhead paddle shape and provide additional areas through which to deliver energy so as to create a more solid lesion. Again, in this embodiment, the electrode 1030 resides in a plane aligned with a longitudinal axis 1010 of the shaft 1020, and the electrode 1030 is comprised of a flexible material which allows the electrode 1030 to bend into a variety of planes relative to the longitudinal axis 1010 including perpendicular to the longitudinal axis 1010. In this embodiment, such bending is achieved with the use of a pullwire 1050 extending at least partially through the shaft 1020 and connected with a portion of the electrode 1030. Pulling the pullwire 1050, as illustrated in FIG. 26B, bends the electrode 1030 into a plane that is at an angle to the longitudinal axis 1010. Thus, the electrode 1030 is able to be desirably arranged prior to contact with the target tissue. This separates positioning from contact force applied during delivery of energy.

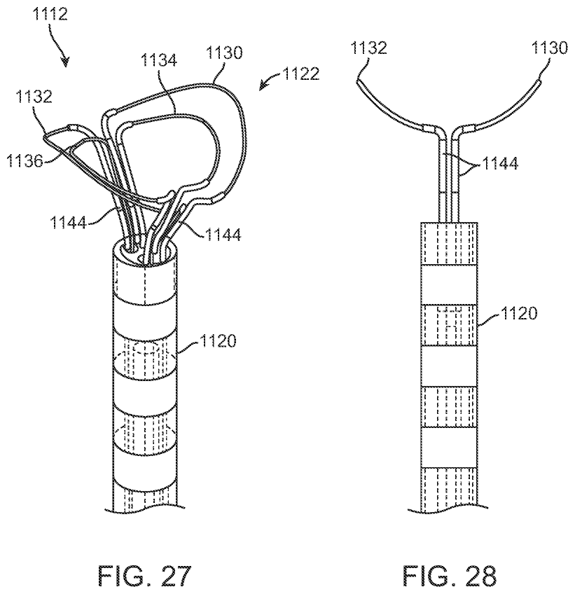

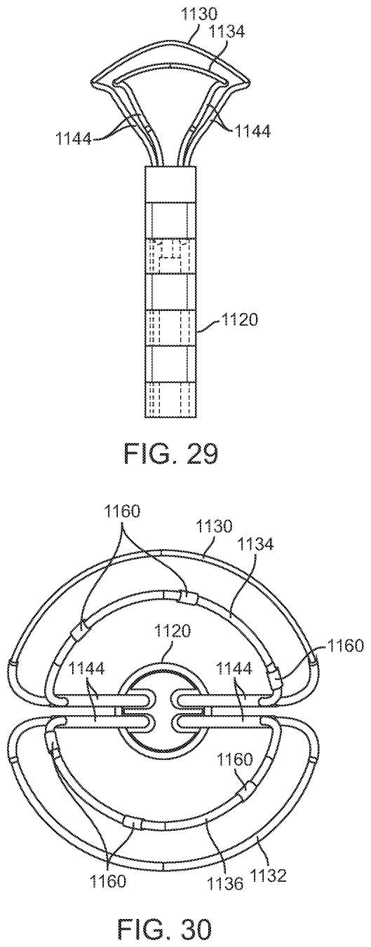

FIGS. 27-30 illustrates another embodiment of a treatment catheter 1112. This embodiment is primarily configured for focal delivery of energy to tissue surfaces, such as in a point-by-point fashion. This is due to the shape and configuration of its delivery electrode 1122. FIG. 27 illustrates an embodiment of the treatment catheter 1112 wherein the delivery electrode 1122 comprises two semi-circular loop electrodes 1130, 1132 which together form a circular or oval shape which is perpendicular to the shaft 1120. The electrodes 1130, 1132 are connected to the shaft 1120 by supports 1144 which are typically insulated so as to direct the energy to the semi-circular electrodes 1130, 1132. In this embodiment, the delivery electrode 1122 further includes an additional set of two semi-circular inner loops 1134, 1136 which together form a circular or oval shape having a small diameter than the two semi-circular loop electrodes 1130, 1132. Thus, the two semi-circular inner loops 1134, 1136 reside “inside” the two semi-circular loop electrodes 1130, 1132, both of which reside on planes which are substantially perpendicular the shaft 1120. In this embodiment, the two semi-circular loop electrodes 1130, 1132 and the two semi-circular inner loops 1134, 1136 have an overall cupped or concave shape. FIG. 28 provides a side view of the embodiment of FIG. 27 which illustrates the cupped shape. Likewise, FIG. 29 provides another side view which illustrates the position of the inner loops 1134, 1136 and loop electrodes 1130, 1132 relative to each other. Thus, the inner loops 1134, 1136 and loop electrodes 1130, 1132 arc in the distal direction so that advancement of the catheter 1112 toward a target tissue location allows the loop electrodes 1130, 1132 to contact the tissue first followed by the inner loops 1134, 1136. This ensures contact of the loop electrodes 1130, 1132 with the tissue. In addition, in some embodiments the inner loops 1134, 1136 provide additional stability. For example, in some embodiments the loop electrodes 1130, 1132 are comprised of a flexible material that allows the loop electrodes 1130, 1132 to bend proximally, so as to form a less concave shape (e.g. a more shallow cupped shape, a more flattened shape or a more convex shape) and conform to the target tissue area. In some embodiments, the inner loops 1134, 1136 are comprised of a stiffer material than the loop electrodes 1130, 1132 so as to act as an anchor during placement, providing more confidence to the user. In addition, the stiffer material resists bending more than the loop electrodes 1130, 1132 which limits the bending of the inner loop electrodes 1130, 1132. In some instances, this is beneficial in avoiding perforation of tissue.

FIG. 30 illustrates an end view of the delivery electrode 1122 of the embodiment of FIGS. 27-29. As shown, the loop electrodes 1130, 1132 are insulated along the supports 1144 and exposed along the outer rim of the overall circular shape. In this embodiment, the inner loops 1134, 1136 include a plurality of microsensors 1160 spaced along the rims of the overall circular shape. In some embodiments, the microsensors 1160 are configured for contact feedback, visualization under fluoroscopy or sensing for electroanatomic mapping systems. In other embodiments, the microsensors 1160 function as electrodes so as to deliver energy in the formation of a lesion. It may be appreciated that in some embodiments, the functionality of the microsensors 1160 switch back and forth between various options, such as delivering energy during delivery of energy through the loop electrodes 1130, 1132 and sensing between periods of energy delivery. It may also be appreciated that in some embodiments the inner loops 1134, 1136 are comprised of continuous conductive wires so as to provide energy delivery in a manner similar to the loop electrodes 1130, 1132.

It may be appreciated that any suitable number of loop electrodes 1130, 1132 may be present, typically one, two, three, four, five, six, seven, eight or more. It may also be appreciated that the loop electrodes 1130, 1132 may be independently activated, activated together or activated in any combination, such as in pairs, groups or in a sequential pattern of individual or grouped electrodes. Likewise, any suitable number of inner loops 1134, 1136 may be present, typically one, two, three, four, five, six, seven, eight or more. Further, any suitable number of microsensors 1160 may be present, such as one, two, three, four, five, six, seven, eight, nine, ten or more. It may be appreciated that the inner loops 1134, 1136 and/or microsensors 1160 may be independently activated, activated together or activated in any combination, such as in pairs, groups or in a sequential pattern of individual or groups. In some instances, energy delivery via the inner loops 1134, 1136 assists in generating solid lesions rather than donut shaped lesions. Further, it may be appreciated that loop electrodes 1130, 1132 and inner loops 1134, 1136 may be independently activated, activated together or activated in any combination, such as in pairs, groups or in a sequential pattern of individual or grouped electrodes. It may also be appreciated that the loop electrodes 1130, 1132 may include microelectrodes through which energy is delivered rather than via a conductive wire.

As mentioned, the delivery electrode 1122 is typically sized in configured to deliver focal energy. In such embodiments, the loop electrodes 1130, 1132 form a circular shape having a diameter in the range of 8 to 14 mm. In other embodiments, the delivery electrode 1122 is configured to provide one-shot delivery. In such embodiments, the loop electrodes 1130, 1132 form a circular shape having a diameter in the range of 22 to 33 mm.

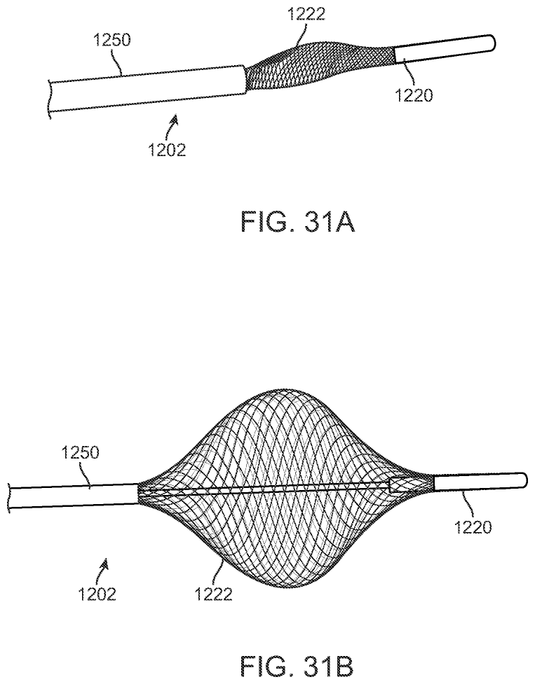

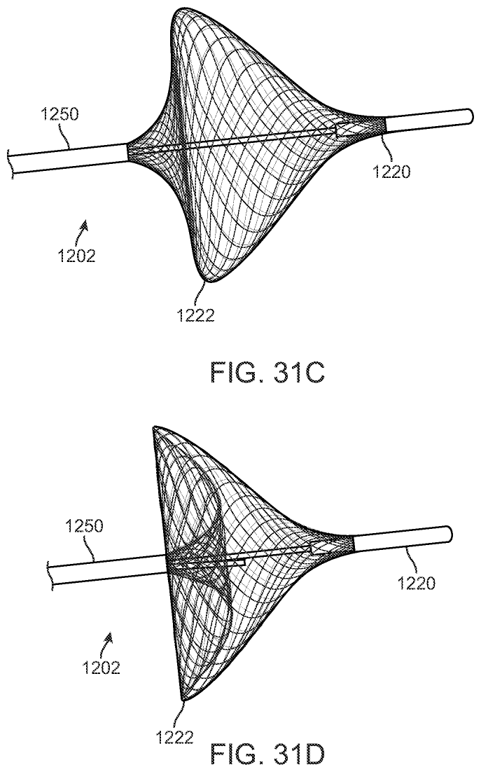

FIGS. 31A-31D illustrate an embodiment of a treatment catheter 1202 configured for one shot delivery rather than focal delivery. Here, the treatment catheter 1202 includes a shaft 1220 that extends into a lumen, such as a pulmonary vein, during placement of the delivery electrode 1222. Given this arrangement, the delivery electrode 1222 is configured to provide energy circumferentially around the lumen, either interiorly, exteriorly or both, with “one-shot” of energy delivery. It may be appreciated that this may be repeated as desired.

In this embodiment, the energy delivery body or delivery electrode 1222 comprises a mesh basket that is configured to move at least between a collapsed configuration, an expanded configuration and a partially inverted configuration. FIG. 31A illustrates the delivery electrode 1222 collapsed around the shaft 1220 upon which it is mounted so that it is able to be housed within a sheath 1250. FIG. 31B illustrates the delivery electrode 1222 in an expanded configuration. This may be achieved by retracting the sheath 1250 or advancing the shaft 1220. In some embodiments, the delivery electrode 1222 is self-expanding upon release from the sheath 1250. In other embodiments, the mesh basket is coupled with an additional shaft that is movable in relation to the shaft 1220 wherein advancement of the additional shaft expands the mesh basket.

In some embodiments, the mesh basket is comprised of a plurality of wires that are energizable in unison, such as to provide energy delivery in a monopolar fashion. It may be appreciated that portions of the mesh basket may be insulated so as to focus energy delivery through particular uninsulated portions of the mesh basket. This may also assist in reducing loss of energy to the surrounding blood environment. In other embodiments, the plurality of wires are independently energizable or energizable in groups. This may also be used to provide energy delivery in a monopolar fashion or it may be used to deliver energy in a bipolar fashion.

The energy delivery electrode 1222 may be utilized in this configuration to deliver energy. For example, the delivery electrode 1222 may be positioned within a lumen so as to deliver energy circumferentially to the inner surfaces of the lumen. Or the delivery electrode 1222 may be positioned against the opening of a lumen so that the distal face of the mesh basket delivers energy to the circumference of the opening of the lumen. In either case, the diameter of the delivery electrode 1222 may be chosen or adjusted to desirably fit the anatomy, such as the pulmonary vein of the specific patient, by controlling expansion of the mesh basket. In addition, the flexibility of the mesh basket allows the delivery electrode 1222 to adapt to a range of circular and non-circular shaped lumens or portions of the anatomy.

FIGS. 31C-31D illustrate further manipulation of the delivery electrode 1222 so as to move the delivery electrode 1222 into a partially inverted configuration. In some embodiments, this is achieved by further advancement of the additional shaft (i.e. beyond expansion of the mesh basket) so that the mesh basket begins to buckle as illustrated in FIG. 31C. Further advancement, as illustrated in FIG. 31D partially inverts the mesh basket so that the distal face of the mesh basket maintains a funnel shape while the proximal face of the mesh basket is inverted. Such inversion provides support for the distal face and assists in maintaining the funnel shape, particularly during placement against tissue and delivery of energy.

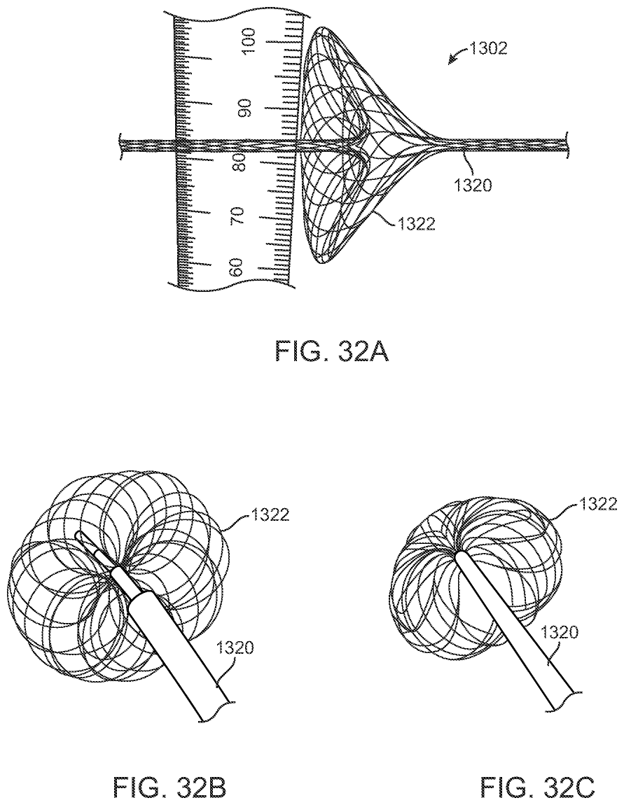

FIGS. 32A-32C illustrate an embodiment of a treatment catheter 1302 similar to that of FIGS. 31A-31D. The treatment catheter 1302 is again configured for one shot delivery rather than focal delivery. And, in this embodiment, the delivery electrode 1322 comprises a wire basket that is somewhat similar to the mesh basket of FIGS. 31A-31D. The wire basket has less surface area exposed to blood and therefore more efficient energy delivery. In some embodiments, the wire basket is comprised of a plurality of wires that are energizable in unison, such as to provide energy delivery in a monopolar fashion. It may be appreciated that portions of the wire basket may be insulated so as to focus energy delivery through particular uninsulated portions of the wire basket. This may also assist in reducing loss of energy to the surrounding blood environment. In other embodiments, the plurality of wires are independently energizable or energizable in groups. This may also be used to provide energy delivery in a monopolar fashion or it may be used to deliver energy in a bipolar fashion.

The treatment catheter 1302 is configured to move at least between a collapsed configuration, an expanded configuration and a partially inverted configuration. FIG. 32A illustrates the delivery electrode 1322 in a partially inverted configuration. FIG. 32B illustrates the delivery electrode 1322 positioned against the opening of a lumen so that the distal face of the wire basket delivers energy to the circumference of the opening of the lumen. FIG. 32C illustrates the delivery electrode 1322 advanced into the lumen so that the distal face of the delivery electrode 1322 is positioned at least partially within the lumen.

Again, the diameter of the delivery electrode 1322 may be chosen or adjusted to desirably fit the anatomy, such as the pulmonary vein of the specific patient, by controlling expansion of the wire basket. In addition, the flexibility of the mesh basket allows the delivery electrode 1322 to adapt to a range of circular and non-circular shaped lumens or portions of the anatomy.

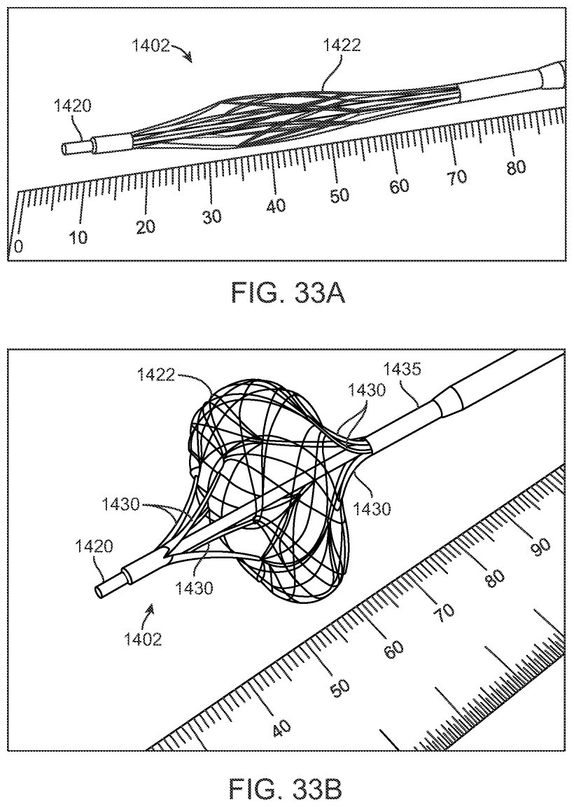

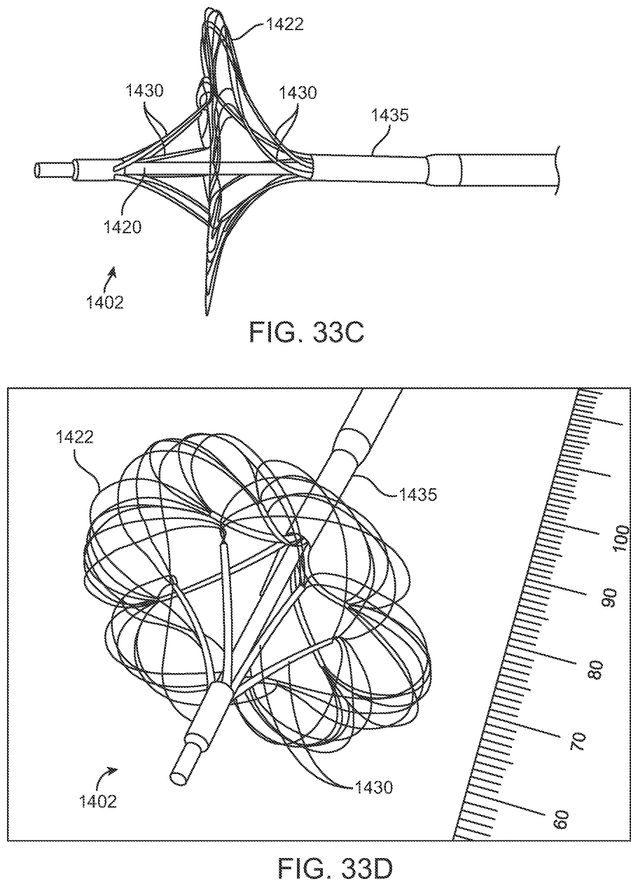

FIGS. 33A-33D illustrate an embodiment of a treatment catheter 1402 similar to that of FIGS. 31A-31D and FIGS. 32A-32C. The treatment catheter 1402 is again configured for one shot delivery rather than focal delivery. And, in this embodiment, the delivery electrode 1422 comprises a wire basket that is somewhat similar to the mesh basket of FIGS. 31A-31D and wire basket of FIGS. 32A-32C. Here, the wire basket has even less surface area exposed to blood and therefore provides more efficient energy delivery. This is achieved by the presence of legs or common supports 1430 on the distal and proximal faces of the wire basket, rather than woven wires. Since these portions typically align with the lumen, contact area is not needed and energy can be focused to the woven portion of the wire basket.

In some embodiments, the wire basket is comprised of a plurality of wires that are energizable in unison, such as to provide energy delivery in a monopolar fashion. It may be appreciated that portions of the wire basket may be insulated so as to focus energy delivery through particular uninsulated portions of the wire basket. This may also assist in reducing loss of energy to the surrounding blood environment. In other embodiments, the plurality of wires are independently energizable or energizable in groups. This may also be used to provide energy delivery in a monopolar fashion or it may be used to deliver energy in a bipolar fashion.

The treatment catheter 1402 is configured to move at least between a collapsed configuration, an expanded configuration and a flattened configuration. FIG. 33A illustrates the delivery electrode 1422 collapsed around the shaft 1420 upon which it is mounted so that it is able to be housed within a sheath (not shown). FIG. 33B illustrates the delivery electrode 1422 in an expanded configuration. This may be achieved by retracting the sheath or advancing the shaft 1420 so as the reveal the wire basket. In some embodiments, the delivery electrode 1422 is self-expanding upon release from the sheath. In other embodiments, the wire basket is coupled with an additional shaft 1435 that is movable in relation to the shaft 1420 wherein advancement of the additional shaft 1435 draws the supports 1430 toward each other, expanding the wire basket.

The energy delivery electrode 1422 may be utilized in this configuration to deliver energy. For example, the delivery electrode 1422 may be positioned within a lumen so as to deliver energy circumferentially to the inner surfaces of the lumen. Or the delivery electrode 1422 may be positioned against the opening of a lumen so that the distal face of the wire basket delivers energy to the circumference of the opening of the lumen. In either case, the diameter of the delivery electrode 1422 may be chosen or adjusted to desirably fit the anatomy, such as the pulmonary vein of the specific patient, by controlling expansion of the wire basket. In addition, the flexibility of the wire basket allows the delivery electrode 1422 to adapt to a range of circular and non-circular shaped lumens or portions of the anatomy.

FIGS. 33C-33D illustrate further advancement of the additional shaft 1435 so as to move the delivery electrode 1422 into a flattened configuration. Here the supports 1430 are fully drawn together so as to flatten the wire basket therebetween substantially into a plane perpendicular to the shaft 1420, as illustrated in FIG. 33C. FIG. 33D provides a perspective view of the configuration of the wire basket depicted in FIG. 33C. Here it can be seen that the wire basket forms loops that extend radially outwardly away from the supports 1430. The loops are able to deliver energy across a larger area than a single wire. The configuration of the loops are optimized to provide maximum coverage in an expanded position while still being able to be collapsed against the shaft for delivery through a small introducer lumen.

It may be appreciated that the delivery electrode 1422 may be positioned against the opening of a lumen, such as a pulmonary vein, such as in a manner as illustrated in FIG. 32B. Likewise, the flexibility of the delivery electrode 1422 allows the delivery electrode 1422 to be advanced into the lumen so that the distal face of the delivery electrode 1422 is positioned at least partially within the lumen, such as in a manner as illustrated in FIG. 32C.

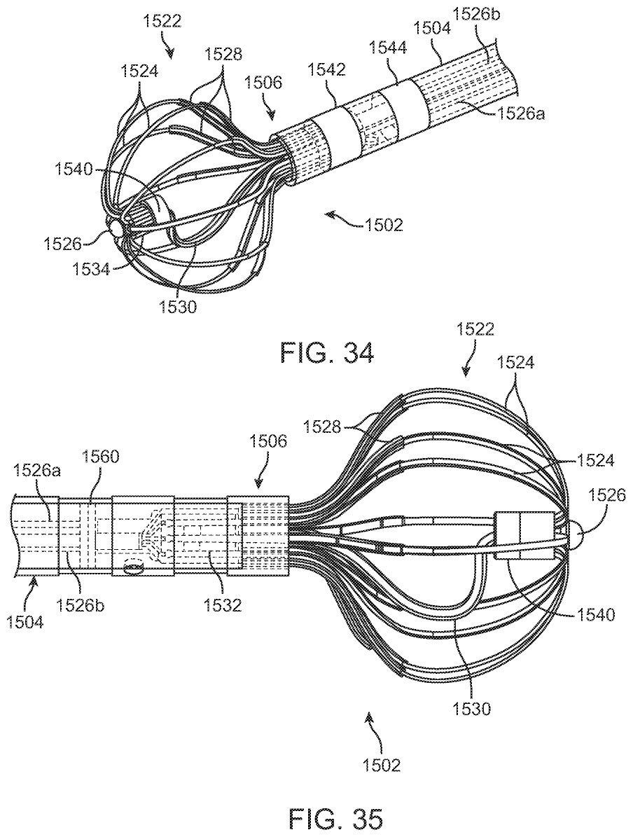

FIGS. 34-38, FIGS. 39A-39B illustrate an embodiment of a treatment catheter 1502 configured for focal delivery. Here, the treatment catheter 1502 comprises a shaft 1504 having a distal end 1506 and an energy delivery body 1522 disposed near the distal end 1506. Here, the energy delivery body 1522 comprises a plurality of conductive splines 1524 forming a convex distal face. In addition, in this embodiment, the energy delivery body 1522 includes a distal tip electrode 1526. Here, the distal tip electrode 1526 is disposed along the center of the convex distal face. This provides additional energy delivery to the tissue upon which the convex distal face is positioned. This assists in avoiding any potential low or missing area of energy delivery, such as would generate a donut shaped lesion in the tissue. Thus, a continuous, circular lesion is created.

It may be appreciated that each spline 1524 may act as an electrode wherein the splines are energized in unison, independently or in groups. Thus, in some embodiments, the energy is delivered from all or a subset of the plurality of splines 1524 in unison so that the energy delivery body 1522 delivers energy in a monopolar fashion with the use of at least one remote return electrode. Likewise, the distal tip electrode 1526 may additionally be energized in unison with the splines 1524 so as to deliver energy is unison in a monopolar fashion. In other embodiments, the energy is delivered between selected splines or selected groups of splines so that the energy is delivered in a bipolar fashion. Likewise, combinations of one or more splines 1524 and the distal tip electrode 1526 may be energized to deliver energy in a bipolar fashion. Further, energy may be delivered from the tip electrode 1526 alone, without energy delivery from one or more splines 1524, and vice versa wherein energy is delivered from one or more splines 1524 and not from the tip electrode 1526. It may be appreciated that the splines 1524 may be wires, flat wires, struts, planks, strips or the like. In this embodiment, the splines 1524 are comprised of shape memory material, such as nitinol flat wire. In this embodiment, the nitinol flat wire has a platinum core for improved visualization under fluoroscopy. In this embodiment, the plurality of splines 1522 are partially covered by insulative material 1528. Here, the insulative material 1528 is located on the proximal side of the energy delivery body 1522 so that energy conducted to the plurality of splines 1524 resists delivery through the insulative material 1528, directing the energy to the uninsulated portion of the plurality of splines 1522 facing distally. Consequently, the energy provided to the energy delivery body 1522 is focused in the distal direction. Since the distal convex face is positionable against the target tissue area, the energy is efficiently directed toward the target tissue area without loss of energy out the proximal side of the energy delivery body 1522. This conserves energy and reduces energy sink to surrounding blood, etc.

In this embodiment, the distal tip electrode 1526 is comprised of platinum-iridium and has a ball shape. It may be appreciated that other suitable materials may be used and other shapes may be used, such as a flat shape, oblong shape or a pointed shape, to name a few. In some embodiments, the distal tip electrode 1526 facilitates directing the treatment catheter 1502 to the target tissue area. This is achieved by using the distal tip electrode 1526 to detect areas of active cardiac tissue that are still needing to be treated. Thus, by reading the electrogram, the next placement of the catheter can be determined. In some embodiments, the distal tip electrode 1526 is used for recording data as will be described in later sections.

The energy delivery body 1522 is transitionable between a collapsed configuration and an expanded configuration. FIG. 34 illustrates the energy delivery body 1522 in an expanded configuration. To collapse the energy delivery body 1522, a sheath or delivery tube is advanceable over the shaft 1504 in the distal direction. As the sheath is advanced over the energy delivery body 1522, the flexibility of the splines 1524 allow the splines 1524 to straighten, thereby causing the profile of the energy delivery body 1522 to flatten and fit within the sheath. In addition, it may be appreciated that such straightening of the splines 1524 lengthens the distance between the distal tip electrode 1526 and the distal end 1506 of the shaft 1504. For this reason, a tip electrode wire 1530 that conducts energy through the shaft 1504 to the distal tip electrode 1526 is slacked when the energy delivery body 1522 is in the expanded configuration as shown in FIG. 34. Such slack allows for lengthening when in the collapsed configuration.

It may be appreciated that the catheter 1504 is delivered to the target treatment area within the body while the energy delivery body 1522 is collapsed and held within a sheath, sleeve or delivery device. Upon desired positioning in the body, the energy delivery body 1522 is then advanced from the sheath (or the sheath is retracted) so that the energy delivery body 1522 is exposed. Such exposure allows the energy delivery body 1522 to self-expand to the expanded configuration. It may be appreciated that in other embodiments, the energy delivery body 1522 may be expanded by other mechanism, such as by retraction of a plunger connected with the distal tip electrode 1526 or by expansion of a flexible expandable member (e.g., a balloon) within the energy delivery body 1522. However, the embodiment of FIG. 34 provides an energy delivery body 1522 that is free of a central shaft creating a hollow rounded cage. This allows for additional flexibility of the energy delivery body 1522. For example, when pressing the convex distal face against target tissue, additional force may allow the plurality of splines 1524 to flex outwardly, increasing the diameter of the convex distal face. Likewise, movement of the shaft 1504 while keeping the convex distal face stationary, may allow increased force against the tissue in the direction of movement due to flexing of the splines 1524. For example, movement of the shaft 1504 to the right during engagement may increase engagement of the splines 1524 on the right side of the convex distal face and allow more force against this area of the tissue. In addition, such increased flexibility may also allow the energy delivery body 1522 to be steered more easily, such as to bend more freely with the use of pullwires or the like.

FIG. 35 provides a side view of the embodiment of the treatment catheter 1502 of FIG. 34. Again, the plurality of splines 1524 are illustrated in an expanded configuration wherein the energy delivery body 1522 forms a ball-shaped cage having a convex distal face. In this embodiment, the plurality of splines 1524 come together, spaced around a shaft plug 1532 within the shaft 1504, in an evenly spaced circumferential array. In this embodiment, each of the splines 1524 are connected to a conductive wire that extends through the shaft 1504 for connection with the energy generator. In this embodiment, the plurality of splines 1524 terminate around the distal tip electrode 1526 by curving and bending inward for mounting on a tip inner 1534, as can be seen by referring back to FIG. 34. Thus, in this embodiment, the splines 1524 are evenly spaced in a circumferential array around the tip inner 1534. By curving and bending inward around the distal tip electrode 1526, a smooth, distal face is formed for positioning against the target tissue.

In this embodiment, the energy delivery body 1522 includes a sensing electrode 1540 positioned within the energy delivery body 1522 so as to avoid contact with the target tissue. In this embodiment, the sensing electrode 1540 is disposed proximally of the distal tip electrode 1526 (i.e. behind the distal tip electrode 1526) within the rounded cage of the plurality of splines 1524. Here, the sensing electrode 1540 comprises a ring electrode, such as a single 0.030″ ring electrode, extending around the tip inner 1534. In this embodiment, additional electrodes 1542, 1544 are disposed along the shaft 1504, such as two 0.070″ ring electrodes, proximal to the energy delivery body 1522. In this embodiment, the electrodes 1540, 1542, 1544 are comprised of platinum-iridium and also serve as marker bands for visualization under fluroscopy.

The sensing electrode 1540 and the additional electrodes 1542, 1544 are typically used for sensing ECG signals and also for providing information to an electroanatomic mapping system. For example, when sensing ECG signals, a user can verify or confirm location of the treatment catheter 1502 in the heart based on the sensed ECG signals. When approaching a ventricle, a user may verify such approach by checking that ventricular signals increase in amplitude. Likewise, in some instances, impedance measurements are tracked by the sensing electrode 1540 and/or the additional electrodes 1542, 1544. Electroanatomic mapping systems use such impedance measurements to visualize the location of these electrodes 1540, 1542, 1544, and therefore the location of the treatment catheter 1502, within the heart.

Referring to FIG. 35, this embodiment also includes a steering mechanism. In this embodiment, the steering mechanism comprises a pullring 1560 disposed along the shaft 1504. The pullring 1560 is connected with one or more pullwires. In this embodiment, the pullring 1560 is connected to a first pullwire 1562a and a second pullwire 1562b, wherein the pullwires 1562a, 1562b are attached to the pullring 1560 on opposite sides. The pullwires 1562a, 1562b extend toward the proximal end of the shaft 1504 so that the distal end 1506 can be manipulated remotely. Pulling of the first pullwire 1562a causes the distal end 1506 and therefore energy delivery body 1522 to bend in the direction of the first pullwire 1562a (e.g. to the left) and pulling of the second pullwire 1562b causes the distal end 1506 and therefore energy delivery body 1522 to bend in the direction of the second pullwire 1562b (e.g. to the right). It may be appreciated that any suitable number of pullwires may be present to steer in a variety of directions. Likewise, other steering mechanisms may be used instead of or addition to the steering mechanisms described herein.

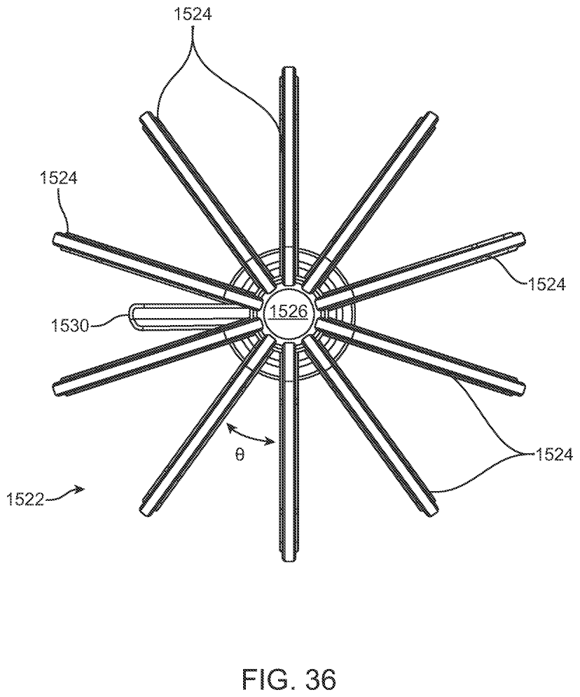

FIG. 36 illustrates a bottom view of the treatment catheter 1502 of FIGS. 34-35, facing the convex distal face of the energy delivery body 1522. As illustrated, in this embodiment, the energy delivery body 1522 includes ten splines 1524, however any suitable number of splines 1524 may be present including one, two, three, four, five, six, seven, eight, nine, ten, eleven, twelve or more. Here, a distal portion of each spline 1524 extends radially outwardly from the distal tip electrode 1526 before curving back toward the proximal direction to form a ball, sphere or rounded cage shape. Thus, each of these spline surfaces are uninsulated and deliver energy to the tissue. In this embodiment, an angle θ forms between each spline 1524, therefore each angle is 36 degrees. It may be appreciated that the angles θ will vary depending on the number of splines 1524, however such angles θ are typically in a range of 10-45 degrees, such as 10-20 degrees, 20-30 degrees or 30-45 degrees. With fewer splines 1524, such angles θ may increase to 50 degrees, 60 degrees, 70 degrees, 80 degrees or 90 degrees, to name a few.

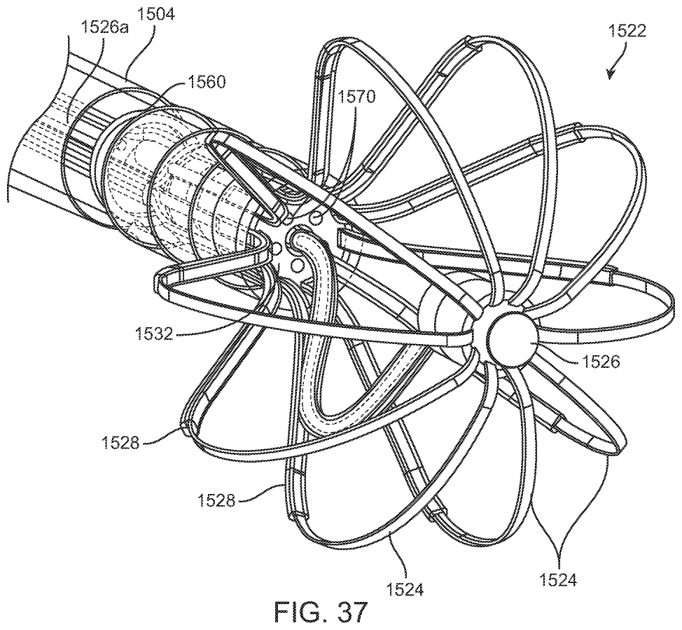

FIG. 37 provides another perspective view of the embodiment of FIG. 34. In this view, irrigation ports 1570 are visible along the distal face of the shaft plug 1532. The irrigation ports 1570 are so positioned so as to deliver irrigation fluid to a proximal end of the energy delivery body 1522 allowing flow toward a distal end of the energy delivery body 1522 (i.e. toward the tip inner 1534 and distal tip electrode 1526). Such irrigation assists in reducing any potential blood clot formation along the energy delivery body 1522. In some instances, blood clotting may be more likely between elements such as splines 1524 which are closely spaced and are positioned in a blood-filled field. As illustrated in FIG. 37, the distance between the splines 1524 tapers toward the proximal end of the energy delivery body 1522 and toward the distal end of the energy delivery body 1522. Such areas are more prone to blood clotting due to increased blood stagnation in these areas. Stagnant blood may clot causing risks to the patient. Using a flow of irrigation fluid, such as saline, in and around the splines 1524 decreases the likelihood of clots forming.

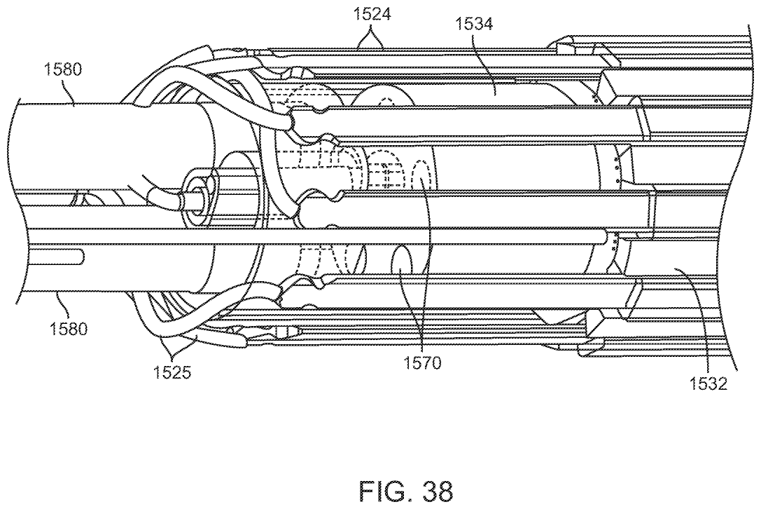

It may be appreciated that desired irrigation fluid flow would be sufficient to reach all or a majority of the potentially stagnant blood-filled areas around the splines 1524. In some embodiments, this is achieved with the use of a plurality of irrigation ports 1570 that are configured to create turbulent flow within the hollow cage of the energy delivery body 1522. Although a single irrigation lumen may provide a large enough output of fluid flow to reach the proximal end of the energy delivery body 1522, the flow may not be strong enough to reach the distal end of the energy delivery body 1522. However, having the single irrigation lumen pass fluid through multiple irrigation ports 1570 creates turbulence in the flow at the proximal end of the energy delivery body 1522 and the turbulent flow continues a wide fan of fluid flow through to the distal end of the energy delivery body 1522. The wide fan also accounts for coverage of the energy delivery body 1522 when the energy delivery body 1522 has been bent or moved laterally during positioning or manipulation. It may be appreciated that such turbulent flow may also be achieve with the use of multiple irrigation lumens. Typically, the number of irrigation lumens is less than the number of irrigation ports so as to deliver adequate flow while creating turbulence. FIG. 38 provides a close-up illustration of a portion of the treatment catheter 1502 within the distal end 1506 of the shaft 1504. Here, the shaft 1504 is removed to reveal the shaft plug 1532 with splines 1524 disposed therearound. Conduction wires 1525 are shown connected with each spline 1525. The conduction wires 1525 extend along the shaft 1504 in the proximal direction for connection with the generator 108 so as to deliver the energy to the splines 1525. The embodiment of FIG. 38 includes two irrigation lumens 1580 that deliver fluid to the irrigation ports 1570. Here, five irrigation ports 1570 are present. It may be appreciated that a variety of irrigation lumens 1580 may be present, including one, two, three, four, five, six or more. Likewise, a variety of irrigation ports 1570 may be present, including one, two, three, four, five, six, seven, eight, nine, ten, eleven, twelve or more. However, for turbulent flow, the irrigation ports 1570 typically outnumber the irrigation lumens 1580.

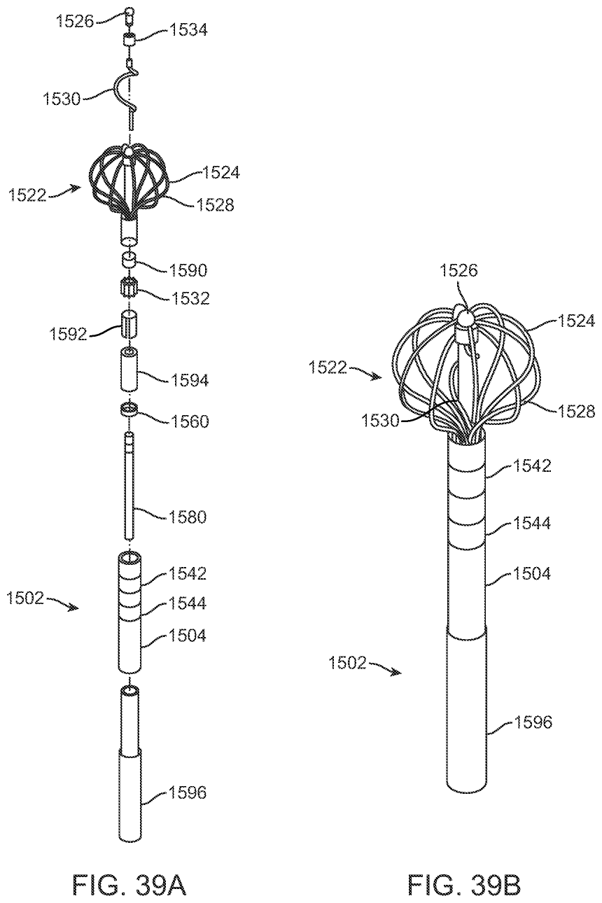

FIGS. 39A-39B provide additional illustrations of the embodiment of FIG. 34. FIG. 39A provides an expanded illustration of elements comprising this embodiment of the treatment catheter 1502. As shown, this embodiment includes a distal tip electrode 1526, a tip inner 1534, a tip electrode wire 1530, an energy delivery body 1522 comprising a plurality of splines 1524 at least partially covered by insulative material 1528, a retention band 1590, a shaft plug 1532, a solder plate 1592, a glue potting 1594, a pull-ring 1560, an irrigation lumen 1580, a shaft 1504 with electrodes 1542, 1544, and a shaft tip section 1596. FIG. 39B illustrates the treatment catheter 1502 of FIG. 39A in its unexpanded state.

As mentioned previously, the treatment catheter 1502 is described as a focal therapy device designed to create lesions that are larger than the footprint of a solid tipped treatment catheter, such as illustrated in FIGS. 2A-2B, yet smaller than the footprint of a one-shot device. In some embodiments, the shaft 1504 of the treatment catheter 1502 is 8 French (2.67 mm) and is delivered with the use of an 8.5 French (2.83 mm) sheath. In such embodiments, the energy delivery body 1522 is configured to fit within the 8.5 French sheath in its collapsed configuration, thus having an outer diameter of less than 2.83 mm. When the energy delivery body 1522 is released to its expanded state, the outer diameter typically expands to 8-15 mm which is 3-6 times the diameter of the shaft 1504. It may be appreciated that the footprints of such devices may vary in size within this range including 8 mm, 9 mm, 10 mm, 11 mm, 12 mm, 13 mm, 14 mm, 15 mm, 8-10 mm, 9-10 mm, 9-15 mm, 10-15 mm, 12-15 mm, etc.

### Sensors and Irrigation

The tissue modification systems 100 described herein deliver a series of PEF batches or bundles described herein over a period of time, such as several seconds. This accumulation of energy deposition results in a small amount of joule heating which is inherent to all PEF therapies as it is a byproduct of energy deposition. However, acute, subacute, medium-, and long-term histological data all indicate that there are no substantial indication of thermal damage to the tissue using the systems, devices and methods described herein. Therefore, it is evident that thermal damage (extracellular protein denaturation) is not generated in the cardiac tissue, reducing the chances of adverse events and anatomical deficits such as pulmonary vein stenosis resulting from the treatment. This also eliminates the generation of surface char or thermal injury which impedes energy delivery to underlying tissue, reducing the ability to generate transmural lesions.

However, it may be appreciated that, in some embodiments, the system 100 includes temperature sensing and/or control measures for various purposes. In some embodiments, temperature is sensed and controlled to ensure that the temperature remains in the range of 30-65° C., 30-60° C., 30-55° C., 30-50° C., 30-45° C., 30-35° C. Thus, lesions are not created by thermal injury as the temperature of the tissue remains below a threshold for thermal ablation. In some embodiments, one or more temperature sensors are used to measure electrode and/or tissue temperature during treatment to ensure that energy deposited in the tissue does not result in any clinically significant tissue heating. For example, in some embodiments, a temperature sensor monitors the temperature of the tissue and/or electrode, and if a pre-defined threshold temperature is exceeded (e.g. 65° C.), the generator alters the algorithm to automatically cease energy delivery or modifies the algorithm to reduce temperature to below the pre-set threshold. For example, in some embodiments, if the temperature exceeds 65° C., the generator reduces the pulse width or increases the time between pulses and/or packets (e.g. delivering energy every other heart beat, every third heart beat, etc.) in an effort to reduce the temperature. This can occur in a pre-defined step-wise approach, as a percentage of the parameter, or by other methods. It may be appreciated that temperature sensors may be positioned on electrodes, adjacent to electrodes, or in any suitable location along the distal portion of the catheter. Alternatively or in addition, sensors may be positioned on one or more separate instruments.

In other embodiments, temperature is sensed to assess lesion formation. This may be particularly useful when generating lesions in anatomy having target tissue areas of differing thicknesses. A rapid rise in temperature indicates that the lesion has penetrated the depth of the tissue and is nearing completion. Sensing such changes in temperature may be particularly useful when generating lesions in thicker tissues or tissues of unknown depth.

In some embodiments, the treatment catheter includes irrigation to assist in controlling the temperature of the delivery electrode or surrounding tissue. In some instances, irrigation cools the delivery electrode, allowing more PEF delivery per time without increasing any potential heat-mediated damage. In some instances, irrigation also reduces or prevents coagulation near the tip of the catheter. It may be appreciated that irrigation may be activated, increased, reduced or halted based on information from one or more sensors, particularly one or more temperature sensors.

Such cooling is achieved by delivering fluid, such as isotonic saline solution, through a lumen in the catheter that exits through one or more irrigation ports along the distal end of the catheter. The fluid may be chilled fluid, room temperature fluid or warmed fluid. The fluid flow can be driven by a variety of mechanisms including a gravity driven drip, a peristaltic pump, a centrifugal pump, etc. In some embodiments, the irrigation has a flow rate of 0.1-10 ml/min, including 1 ml/min, 2 ml/min, 3 ml/min, 4 ml/min, 5 ml/min or more. In some embodiments, the flow rate is sensed by electrical or mechanical flow sensing mechanisms. In some embodiments, the temperature of the fluid is measured, and in other embodiments the temperature of the fluid is modified, such as warmed or cooled, as it is pumped into the treatment catheter, such as based on the measured temperature. In some embodiments, the fluid flow rate is determined based on the measured temperature of the tissue to be treated.

In some embodiments, the pump is in electrical communication with the generator 108 wherein the fluid flow rate is modified by the generator 108 based on the status of energy delivery to the treatment catheter 102. For example, in some embodiments, fluid flow rate is increased during energy delivery. Likewise, in some embodiments, fluid flow rate is increased a predetermined amount to time prior to energy delivery and/or at a predetermined time(s) during energy delivery. Alternatively or in addition, fluid flow may be controlled on demand by the user. It may be appreciated that the pump may communicate with the generator 108 to operate at different speeds based on various aspects of the energy delivery algorithm 152. In some embodiments, sensing of flowrate and communication with the generator 108 is used to prevent energy delivery if irrigation is not running. In other embodiments, selection of an energy delivery algorithm 152 in turn selects a fluid flow rate appropriate for the energy delivery algorithm 152. In some embodiments, at least one irrigation port is located along an electrode and/or optionally at least one irrigation port is located along the shaft.

It may be appreciated that any of the catheter designs described herein may include one or more sensors (e.g. microsensors), such as impedance sensors, contact sensors, contact force sensors, electroanatomic mapping sensors, etc. Such sensors may be positioned on electrodes, adjacent to electrodes, or in any suitable location along the distal portion of the catheter. For example, microsensors may be located along one or more loops of a delivery electrode or along a support structure near the delivery electrode. Alternatively or in addition, sensors may be positioned on one or more separate instruments.

It may also be appreciated that although a variety of the delivery electrodes have been described as conductive wires capable of delivering energy therefrom, such designs may utilize individual electrodes (e.g. microelectrodes) spaced along a non-conductive wire. Optionally, such electrodes may be spaced along a conductive wire when the conductive wire is insulated from the electrodes where the electrodes are attached.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for delivering energy to cardiac tissue of a patient comprising:
a shaft having a proximal end and a distal end, wherein the shaft has an outer diameter; and
an energy delivery body disposed along the distal end of the shaft, wherein the energy delivery body is transitionable between a collapsed configuration and an expanded configuration, wherein the expanded configuration has an outer diameter that is less than or equal to 6 times the outer diameter of the shaft, wherein the energy delivery body is configured to be positioned against the cardiac tissue in the expanded configuration so as to deliver energy to the cardiac tissue.

2. A device as in claim 1, wherein the energy is pulsed electric field energy and wherein the device is configured to deliver the pulsed electric field energy to the cardiac tissue.

3. A device as in any of the above claims, wherein the energy delivery body comprises a plurality of wires configured to deliver the energy.

4. A device as in claim 3, wherein the plurality of wires comprises a plurality of splines.

5. A device as any of claims 3-4, wherein the plurality of wires is comprised of shape memory material so that the energy delivery body is transitionable by release from a sheath that constrains the plurality of wires so that such release allows the plurality of wires to move toward the expanded configuration.

6. A device as in claim 5, wherein the energy delivery body is free of a central shaft when in the expanded configuration.

7. A device as in claim 5, wherein the plurality of splines form a hollow rounded cage.

8. A device as in claim 3, wherein the plurality of wires comprises a mesh.

9. A device as in claim 3, wherein the plurality of wires comprises a plurality of loops.

10. A device as in any of claims 3-9, wherein the plurality of wires is energizable in unison so as to function in a monopolar fashion.

11. A device as in any of claims 3-10, wherein the plurality of wires has a convex distal face.

12. A device as in any of claims 3-11, wherein the energy delivery body includes a distal tip configured to deliver the energy.

13. A device as in claim 12, wherein the distal tip and the plurality of wires are energizable in unison so as to function in a monopolar fashion.

14. A device as in any of claims 3-13, wherein a proximal portion of the plurality of wires is insulated so as to direct the energy toward a distal direction.

15. A device as in any of the above claims, further comprising a plurality of irrigation ports, wherein the device is configured so as to direct fluid through the irrigation ports in a manner that creates turbulent flow of the fluid within the energy delivery body.

16. A device as in claim 15, wherein the plurality of irrigation ports is disposed near a proximal end of the energy delivery body.

17. A device as in any of claims 15-16, further comprising one or more irrigation lumens which direct the fluid through the plurality of irrigation ports.

18. A device as in claim 17, wherein the one or more irrigation lumens is less than the plurality of irrigation ports.

19. A device as in any of the above claims, wherein the expanded configuration has an outer diameter that is 3-6 times the outer diameter of the shaft.

20. A device as in any of the above claims, wherein the expanded configuration has an outer diameter that is 8-15 mm.

21. A device as in any of the above claims, wherein the energy delivery body includes a sensing electrode.

22. A device as in claim 21, wherein the sensing electrode is positioned so as to avoid contact with the cardiac tissue.

23. A device as in claim 22, wherein the energy delivery body comprises a plurality of splines forming a rounded cage and wherein the sensing electrode is disposed within the rounded cage.

24. A device as in any of the above claims, wherein the shaft includes one or more ring electrodes.

25. A device as in any of the above claims, further comprising one or more electrodes that communicate with an electrophysiological mapping system.

26. A device as in any of the above claims, further comprising a steering mechanism configured to bend the energy delivery body in relation to the shaft.

27. A device as in any of the above claims, further comprising a steering mechanism configured to bend the distal end of the shaft away from its longitudinal axis.

28. A device for delivering energy to cardiac tissue of a patient comprising:
a shaft having a proximal end and a distal end; and
an energy delivery body disposed along the distal end of the shaft, wherein the energy delivery body is comprised of a plurality of shape-memory splines and is transitionable between a collapsed configuration and an expanded configuration, wherein in the expanded configuration the plurality of shape-memory splines form a convex distal face positionable against the cardiac tissue so as to deliver energy to the cardiac tissue.

29. A device as in claim 28, wherein the plurality of shape-memory splines form a rounded cage having the convex distal face in the expanded configuration.

30. A device as in claim 29, wherein the rounded cage is supported solely by the plurality of splines.

31. A device as in claim 30, wherein the energy delivery body includes a distal tip electrode and wherein the plurality of splines supports a tip electrode wire extending from the distal tip electrode to the shaft and is otherwise hollow.

32. A device as in any of claims 29-31, wherein the rounded cage is flexible so as to deform upon positioning against the cardiac tissue.

33. A device as in any of claims 29-32, wherein the rounded cage is flexible so as to at least partially flatten upon positioning against the cardiac tissue.

34. A device as in any of claims 28-33, wherein the convex distal face is configured to have a footprint of 8-15 mm when positioned against the cardiac tissue so as to deliver energy to the cardiac tissue.

35. A device as in any of the above claims, wherein the plurality of splines are energizable in unison to function in a monopolar manner.

36. A device as in any of the above claims, wherein the energy delivery body includes a distal tip electrode disposed along the convex distal face.

37. A device as in claim 36, wherein the distal tip electrode is independently energizable.

38. A device as in any of the above claims, wherein at least a portion of the energy delivery body is insulated so as to direct the energy through the convex distal face.

39. A device as in any of the above claims, further comprising at least one irrigation lumen and a plurality of irrigation ports.

40. A device as in claim 39, wherein the at least one irrigation lumen comprises a number of irrigation lumens that is less than plurality of irrigation ports.

41. A device as in claim 29, wherein the energy delivery body is electrically couplable with a generator so as to deliver pulsed electric field energy to the cardiac tissue.

42. A system for delivering energy to cardiac tissue of a patient comprising:
a treatment catheter comprising
a shaft having a proximal end and a distal end, wherein the shaft has an outer diameter, and
an energy delivery body disposed along the distal end of the shaft, wherein the energy delivery body is transitionable between a collapsed configuration and an expanded configuration, wherein the expanded configuration has an outer diameter that is less than or equal to 6 times the outer diameter of the shaft, wherein the energy delivery body is configured to be positioned against the cardiac tissue in the expanded configuration so as to deliver energy to the cardiac tissue; and
a generator electrically couplable to the treatment catheter, wherein the generator includes at least one energy delivery algorithm configured to provide an electric signal of pulsed electric field energy deliverable through the energy delivery body.

43. A system as in claim 42, wherein the pulsed electric field energy is below a threshold for inducing coagulative thermal damage.

44. A system for treating cardiac tissue of a patient comprising:
a treatment device comprising
a shaft having a proximal end and a distal end, and
an energy delivery body disposed along the distal end of the shaft, wherein the energy delivery body is configured to be positioned against the cardiac tissue, and wherein the energy delivery body is electrically couplable with a generator so as to deliver pulsed electric field energy to the cardiac tissue; and
a generator electrically couplable to the treatment catheter, wherein the generator includes at least one energy delivery algorithm configured to provide an electric signal of pulsed electric field energy deliverable through the energy delivery body.

45. A system as in claim 44, wherein the pulsed electric field energy is below a threshold for inducing coagulative thermal damage.

46. A system for delivering energy to cardiac tissue of a patient comprising:
A treatment catheter comprising
a shaft having a proximal end and a distal end, and
an energy delivery body disposed along the distal end of the shaft, wherein the energy delivery body is comprised of a plurality of shape-memory splines and is transitionable between a collapsed configuration and an expanded configuration, wherein in the expanded configuration the plurality of shape-memory splines form a convex distal face positionable against the cardiac tissue so as to deliver energy to the cardiac tissue; and
a generator electrically couplable to the treatment catheter, wherein the generator includes at least one energy delivery algorithm configured to provide an electric signal of pulsed electric field energy deliverable through the energy delivery body.

47. A system as in claim 46, wherein the pulsed electric field energy is below a threshold for inducing coagulative thermal damage.
